(19) United States
(12) Patent Application Publication
(10) Pub. No.: US 2022/0125450 A1
(43) Pub. Date: Apr. 28, 2022

(54) DEVICES AND METHODS FOR ASPIRATION OF THROMBUS

(71) Applicant: Elixir Medical Corporation, Milpitas, CA (US)

(72) Inventors: MOTASIM SIRHAN, Los Altos, CA (US); VINAYAK BHAT, Cupertino, CA (US); BENJAMYN SERNA, Gilroy, CA (US); SAMIT MUSTAFA, San Francisco, CA (US); BRETT CRYER, Pleasanton, CA (US); KIM NGUYEN, Union City, CA (US); JOHN YAN, Los Gatos, CA (US)

(73) Assignee: Elixir Medical Corporation, Milpitas, CA (US)

(21) Appl. No.: 17/568,559

(22) Filed: Jan. 4, 2022

Related U.S. Application Data
(63) Continuation of application No. PCT/US20/42827, filed on Jul. 20, 2020.
(60) Provisional application No. 62/876,376, filed on Jul. 19, 2019.

(51) Int. Cl. A61B 17/221

(52) U.S. Cl. CPC A61B 17/221; A61B 2017/22079

(57) ABSTRACT

An aspiration catheter for removing clot from a blood vessel includes a catheter body having a scaffold extending distally from a distal end of the body. An aspiration lumen runs from the distal end to a proximal end of the body, and a central clot-receiving passage in the scaffold is contiguous with the aspiration lumen of the catheter body. A vacuum-resistant membrane covers the scaffold and establishes a clot aspiration path from a distal end of the scaffold to a proximal end of the aspiration lumen, in the catheter body so that applying a vacuum to the proximal end of the aspiration lumen can draw clot into the central clot-receiving passage. The scaffold may have a comical configuration, a cylindrical configuration, or a combination thereof, and at least a distal portion of the scaffold is radially expandable from a delivery configuration to an extraction configuration.

### CROSS-REFERENCE TO RELATED APPLICATION

This application is a continuation of PCT Application No. PCT/US20/42827 (Attorney Docket No. 32016-719.601), filed Jul. 20, 2020, which claims the benefit of Provisional No. 62/876,376, (Attorney Docket No. 32016-719.101), filed Jul. 19, 2019, the full disclosure of which is incorporated herein by reference.

This application is a U.S. continuation-in-part of U.S. patent application Ser. No. 16/786,736 (Attorney Docket No. 32016-714.306), filed Feb. 10, 2020, which is a continuation of U.S. patent application Ser. No. 16/518,657 (Attorney Docket No. 32016-714.305), filed Jul. 22, 2019, which is a continuation U.S. patent application Ser. No. 16/356,933 (Attorney Docket No. 32016-714.304), filed Mar. 18, 2019, now U.S. Pat. No. 10,383,750, which is a continuation of U.S. patent application Ser. No. 16/039,194 (Attorney Docket No. 32016-714.303), filed Jul. 18, 2018, now U.S. Pat. No. 10,271,976, which is a continuation of U.S. patent application Ser. No. 15/921,508 (Attorney Docket No. 32016-714.302), filed Mar. 14, 2018, now U.S. Pat. No. 10,076,431, which is a continuation of U.S. patent application Ser. No. 15/605,601 (Attorney Docket No. 32016-714.301), filed May 25, 2017, now U.S. Pat. No. 9,943,426, which is a continuation of PCT Application No. PCT/US2017/032748 (Attorney Docket No. 32016-714.601), filed May 15, 2017, which claims the benefit of provisional patent application No. 62/480,121 (Attorney Docket No. 32016-714.106), filed Mar. 31, 2017; 62/430,843 (Attorney Docket No. 32016-714.105), filed Dec. 6, 2016; 62/424,994 (Attorney Docket No. 32016-714.104), filed Nov. 21, 2016; 62/414,593 (Attorney Docket No. 32016-714.103), filed on Oct. 28, 2016; 62/374,689 (Attorney Docket No. 32016-714.102), filed on Aug. 12, 2016; and 62/337,255 (Attorney Docket No. 32016-714.101), filed on May 16, 2016, the full disclosures of which are incorporated herein by reference.

### BACKGROUND OF THE INVENTION

### 1. Field of the Invention

Every year millions of people worldwide suffer strokes caused by blood clots in the brain. Even when not fatal, these clots can lead to severe and permanent disability. Until recently the only means of treating patients presenting with symptoms of an occlusive stroke was pharmaceutical, in which tissue plasminogen activator (tPA) is given to the patient intravenously to dissolve the clot and restore blood flow in the brain. However, since a vascular thrombus (clot) tends to become more fibrous and/or firm up with time, the efficacy window for tPA is just a few hours after the clot first forms. Considering the time involved with recognizing an individual may be having a stroke, transporting them to the hospital, and performing the diagnosis and applying treatment, many patients' clots are too mature to respond to tPA, such that perhaps two thirds of stroke victims were not being significantly helped by pharmaceutical treatment.

Advances in medical technology led to the development of various mechanical thrombectomy techniques, in which the blood clot is physically extracted from the brain. Mechanical thrombectomy has the major advantage over pharmaceutical treatment in that it can remove clots many hours after the efficacy window for pharmaceutical treatment has passed and still provide benefit to the patient.

There are two primary approaches to mechanical thrombectomy, which may be used independently or in combination with each other depending on patient characteristics and physician preference. The first is to use a catheter to apply a vacuum to the clot, in a technique known as direct aspiration. The second is to use a stent retriever to snare and physically pull out the thrombus, optionally in combination with applying a vacuum to the clot through a separate aspiration catheter.

Both mechanical thrombectomy methods have their limitations. While stent retrievers are small and flexible enough to access most clots, their ability to snag and remove a clot varies. In some cases only a portion of the clot can be removed, and debris from the procedure can be released downstream causing secondary occlusions. Stent retrievers can also induce trauma to the vessel as they are dragged proximal pulling the clot with them. The struts of the retriever scrape the endothelium off the vessel walls, creating areas more prone to generating future occlusions. Procedure time is also an issue with stent retrievers, since in addition to delivery and extraction time they typically require a significant time to settle into and secure the clot before the first removal attempt can be made. In an environment of blood-starved brain tissue, the difference in procedure times is very clinically significant on successful outcomes.

The effectiveness of aspiration catheters depends on the ability of the catheter to vacuum the clot through the aspiration lumen of the catheter. Current aspiration catheters are limited in diameter by the size of the introducer sheath and guiding catheter used by the physician to introduce the aspiration catheter into the anatomy. Since most clots tend to be significantly larger than the aspiration catheter size, the small size of conventional aspiration catheters represents a challenge to successful aspiration, due to their inability to fully aspirate the clot on the first vacuum attempt and in the absence of breaking or fragmenting the clot. Current aspiration catheters are also bulky which limits the ability of such catheters to navigate the tortuous anatomy of the brain to reach the common target occluded segments. Such catheters are even less successful in reaching the more distal clots due to the bulky size of these catheters and the very tortuous neurovascular anatomy. Smaller aspiration catheters designed specifically to access the more distal clots often fail to extract the clot due to lack of sufficient suction force at the tip due to the small tip area, and/or because the aspiration lumen of the smaller catheters is too narrow to absorb the clot. Therefore stent retrievers are utilized more often for such distal occluded vessels, alone or in combination with aspiration catheters. Despite the combination use of a stent retriever and aspiration catheters together, the failure to remove the clot completely or partially is still occurs in a significant number of patients and the procedure length of time is extended, potentially compromising the patient brain cells in that occluded area.

What is needed is a device that is capable of reaching clots in the brain in both the proximal and distal neuro anatomy, a device able to remove clots without fragmenting or without substantially fragmenting the clot, a device able to remove clots without causing secondary occlusions, a device able to remove clots reliably without requiring the use of a stent retriever or other supplementary device, a device able to reach the occlusion and retrieve the clots quickly, a device that does not scrape or otherwise induce trauma to the vessel wall at any point during the procedure, a device that is successful in retrieving the clot during the first aspiration attempt, and a device that requires less vacuum pressure to retrieve a clot. The present invention will address at least some of these needs.

### 2. Listing of Background References

Relevant patents and publications include WO1995/31149; US 2008/0086110; and U.S. Pat. No. 5,403,334.

### SUMMARY OF THE INVENTION

In a first aspect of the present invention, an aspiration catheter for removing clot from a blood vessel comprises a catheter body having a proximal end, a distal end, and an aspiration lumen therebetween. A scaffold extends distally from the distal end of the catheter body and has a central clot-receiving passage contiguous with the aspiration lumen of the catheter body. A vacuum-resistant membrane covering the scaffold establishes a clot aspiration path from a distal end of the scaffold to a proximal end of the aspiration lumen in the catheter body so that applying a vacuum to the proximal end of the aspiration lumen can draw clot into the central clot-receiving passage. At least a distal portion of the scaffold is configured to be radially expandable from a delivery configuration to an extraction configuration.

The delivery configuration is typically a low profile configuration to allow advancement through a patient's vasculature, typically the neuro vasculature but optionally in the cardiac and peripheral vasculature as well. The extraction configuration is usually radially expanded or enlarged with an open port or passage at the distal end of the scaffold to engage and collect clot, thrombus, atheroma, and other obstructive material from the blood vessel when vacuum is applied to the aspiration limen. The scaffold provides mechanical support while the vacuum-resistant membrane establishes a vacuum through the scaffold.

In some instances, the scaffold will be at least partially self-expanding, typically being formed in whole or in part from an elastic material, such as a shape or heat memory metal or plastic, e.g. a nickel-titanium alloy. A sheath may be configured to radially constrain such self-expanding distal portions, where translation of the sheath relative to the catheter body releases the constraint and allows the radially expandable distal portion of the scaffold to radially expand. Other forms of constraint, such as constraining hoops, suture loops, dissolvable adhesives, and the like may also be used for deploying self-expanding scaffold.

In other instances, the radially expandable distal portion of the scaffold is configured to be reversibly driven between a radially contracted configuration and a radially expanded configuration. As described below, such mechanism my comprise a rotating coil, a pair of counter-rotating coils, or the like.

The scaffold in its radially expanded configuration may have a substantially cylindrical distal region configured to engage an inner wall of the blood vessel and a tapered transition region disposed between the cylindrical distal region and the distal end of the catheter body. The cylindrical distal region typically has an open distal end configured to direct clot into the central clot-receiving passage when the vacuum is applied to a proximal end of the aspiration lumen. The cylindrical distal region may have a diameter, when expanded, in a range from 2 mm to 6 mm, typically from 2.2 mm to 5.5 mm and length, when expanded, in a range from 1 mm and 150 mm, preferably in a range from 2 mm to 100 mm, more preferably in a range from 3 mm to 50 mm.

Alternatively the radially expanded configuration may have a substantially conical region with a proximally oriented apical opening attached to the distal end of the catheter body and a distally oriented open base configured to engage an inner wall of the blood vessel and direct clot into the central clot-receiving passage when the vacuum is applied to a proximal end of the aspiration lumen. The distally oriented open base may have a diameter, when expanded, in a range from 2 mm to 6 mm, typically from 2.2 mm to 5.5 mm, while the length between the apical end and the open base, when expanded, in a range from 1 mm and 10 mm, preferably in a range from 2 mm to 5 mm, more preferably in a range from 3 mm to 4 mm.

In other instances, the membrane of the aspiration catheter may cover all or a portion of an inner surface of the scaffold. A distal end of the vacuum-resistant membrane may be located proximally of a distal end of the scaffold, leaving an uncovered distal portion of the scaffold. A distal or other portion of the scaffold may be uncovered (not covered by the vacuum-resistant membrane) and configured to do at least one of invaginate the clot, break the clot, and facilitate extraction of the clot.

In still other instances, an open port of the distal tip of the scaffold in its extraction configuration may has an area which is 1.5 to 10 times greater than the open port area when the scaffold is in its delivery configuration. The entire scaffold may comprise an expandable distal segment. The vacuum-resistant membrane may be coupled to at least the distal portion the scaffold. The delivery configuration of the distal portion of scaffold may smaller than the distal end of the catheter body, and an inner surface of the distal portion of scaffold may be coated with a lubricious material.

In further instances, the scaffold in its extraction configuration may be expanded from a size in a range from that of the clot to that of the vessel. A catheter or a wire may be placed to extend through the aspiration lumen to provide retraction or advancement of the sheath to deploy the scaffold to the expanded configuration. The distal portion of the scaffold in said extraction configuration may be configured to engage an inner wall of the blood vessel to substantially prevent blood proximal to the clot from entering the clot aspiration path when said vacuum is applied, or a proximal portion of the scaffold in said extraction configuration may be configured to engage an inner wall of the blood vessel to substantially reduce blood proximal to the clot from entering the clot aspiration path when said vacuum is applied.

In many instances, the scaffold in the extraction configuration is configured to draw the clot into the central clot-receiving passage when distal end of said scaffold is placed proximal to the clot and vacuum is applied. Additionally, the distal portion of the scaffold may be configured to engage and break up clot when said distal portion is expanded to facilitate suction of said clot into the aspiration lumen. For example, the expandable scaffold may comprise one or more features selected from the group consisting of sharp edges, metallic protrusions, fins, hook elements, and slots to improve cutting of or gripping the clot.

In another example, a thrombectomy catheter for removing occlusive material from a blood vessel includes a catheter body and a radially expandable separator scaffold. The catheter body has a proximal end, a distal end, and an aspiration lumen therebetween. The radially expandable separator scaffold extends distally from the distal end of the catheter body and includes helically arranged cutting elements which define a central clot-receiving passage. The separator scaffold may be radially expanded in the blood vessel and rotated and advanced to resect clot. The aspiration lumen of the catheter body and the central clot-receiving passage of the radially expandable separator scaffold are arranged in-line so that clot resected by rotating the separator scaffold may be aspirated into the aspiration lumen of the catheter body by applying a vacuum to a proximal end of the aspiration lumen.

In those examples where the scaffold is configured to be reversibly driven, the radially expandable distal portion of the scaffold may comprises at least a first coil which is configured to be torqued in at least one rotational direction to radially open or close the radially expandable at least distal portion of the scaffold. In such cases, the vacuum-resistant membrane may comprise an expandable sleeve which covers the at least first coil to enclose the central clot-receiving passage to create a continuous vacuum path from the aspiration lumen to a distal end of the radially distal expandable segment. For example, the expandable sleeve may comprise at least one of an elastic section, a folded section, and a furled section. The at least first coil may be configured to be torqued in both rotational directions to radially open and close the radially expandable portion of the scaffold. The cylindrical distal region of the scaffold further may comprises a rotatable inner member, where the first coil is fixed at its proximal end to a distal end of the catheter body and at its distal end to a distal end of the inner member. In this way, rotation of a proximal end of the inner member rotates the distal end of the first coil. In some instances, the cylindrical distal region of the scaffold may further comprises a rotatable outer member, where the first coil is fixed at its proximal end to a distal end of the catheter body and at its distal end to a distal end of the outer member, wherein rotation of a proximal end of the outer member rotates the distal end of the first coil. The scaffold may still further comprises a second coil rotatably and coaxially mounted within the at least first coil, where the at least one coil is fixed at its proximal end to a distal end of the catheter body and at its distal end to a distal end of the second coil and where the first and second coils are wound in opposite helical directions so that rotation of a proximal end of the second coil in a first direction causes both the first and second coils to radially expand.

In such coiled examples, at least one coil may comprise a helically wound elongated member formed from struts joined by crowns in a serpentine pattern, wherein rotation of a proximal end of the at least one coil releases said struts from a crimped configuration to allow the helically wound elongated member to radially expand.

Optionally, even when the scaffold is coiled and configured to be reversibly driven, the aspiration catheter may still comprise a sheath or cap constraining the at least one coil in its crimped configuration. In those examples where the conical region of the scaffold comprises a plurality of struts having proximal ends disposed about the proximally oriented apical opening and distal ends disposed about the distally oriented open base, such struts may be arranged individually with free proximal ends coupled only by the vacuum-resistant membrane. Alternatively, such struts may be interconnected. In other examples, the struts may be arranged in a serpentine pattern with crown regions disposed about both the proximally oriented apical opening and the distally oriented open base. In still other instances, the struts the scaffold may be configured to be reversibly driven ay diverge radially outwardly in the distal direction to define the conical region when unconstrained.

In those examples where the distal portion of the scaffold in its extraction configuration may have a substantially conical region with a distally oriented apical opening attached to the distal end of the catheter body and a proximally oriented open base configured to engage an inner wall of the blood vessel, a scaffold constraint and release mechanism comprising a sheath may be configured to be advanced distally to cover and constrain the struts and retracted proximally to uncover and release the struts to expand radially. Alternatively or additionally, a scaffold constraint and release mechanism comprising a cap covers and constrains the distal ends of the struts in a first position and uncovers and releases the distal ends of the struts in a second position. An alternative scaffold constraint and release mechanism may comprise a length of material attached to an inner member and wrapping around the struts, wherein the inner member is configured to pull the length of material off the struts to allow them to self-expand. A further alternative may comprise a scaffold constraint and release mechanism comprising an inner member, wherein the struts are initially bonded to the inner member with a frangible material that can be mechanically broken to release the struts to self-expand. A still further alternative scaffold constraint and release mechanism may comprise a filament held under tension around the struts, wherein the tension can be released to allow the struts to self-expand. In yet other examples, the struts may be folded entirely inside the aspiration lumen of the catheter body and are configured to be pushed distally to deploy and open.

In another aspect of the present invention, an aspiration catheter for removing clot from a blood vessel, said aspiration catheter comprises a catheter body having a proximal end, a distal end, and an aspiration lumen therebetween. A scaffold extends distally from the distal end of the catheter body and has a central clot-receiving passage contiguous with the aspiration lumen of the catheter body. A membrane covering the scaffold to establish a clot aspiration path from a distal end of the scaffold to a proximal end of the lumen in the catheter body so that applying a vacuum to a proximal end of the aspiration lumen can draw clot into the central clot-receiving passage while substantially preventing blood proximal to the clot from entering the aspiration lumen. At least a proximal portion of the scaffold is radially expandable from a delivery configuration to an extraction configuration. wherein the radially expanded configuration has a substantially conical region with a distally oriented apical opening attached to the distal end of the catheter body and a proximally oriented open base configured to engage an inner wall of the blood vessel and direct clot into the central clot-receiving passage when the vacuum is applied to a proximal end of the aspiration lumen.

In a still further aspect of the present invention, a method for extracting clot from a blood vessel, said method comprises positioning a radially expandable distal portion of an aspiration catheter in a blood vessel proximal to the clot. A distal portion of the aspiration catheter is radially expanded in the blood vessel to form an enlarged central clot-receiving passage through the radially expandable distal portion contiguous with an aspiration lumen in the aspiration catheter. A vacuum is applied to a proximal portion of the aspiration lumen to draw clot from the blood vessel into the radially expandable distal portion of the aspiration catheter, where the radially expandable distal portion of the aspiration catheter comprises a scaffold covered with a vacuum-resistant membrane with sufficient strength to maintain patency of the central clot-receiving passage while applying the vacuum.

In such methods, a distal end of the radially expandable distal portion may engage the clot when the vacuum is applied. Alternatively, a distal end of the radially expandable distal portion may be spaced proximally of the clot when the vacuum is applied. Alternatively or additionally, a distal end of the radially expandable distal portion may engaged against the clot and manipulated to at least partly breakup the clot prior to or while the vacuum is applied. Optionally, a distal end of the radially expandable distal portion may be positioned to inhibit blood located proximally of the distal portion of an aspiration catheter from entering the aspiration lumen.

With further respect to such method, the radially expandable distal portion of the aspiration catheter may be self-expanding and radially expanding the radially expandable distal portion comprises releasing the radially distal expandable segment from a constraining sheath. Often, radially expanding the radially expandable distal portion of the aspiration catheter comprises actuating a structure on the aspiration catheter to open the central clot-receiving passage. For example, the structure may be actuated to radially constrict the radially distal segment of the aspiration catheter in the blood vessel to close central clot-receiving passage. Actuating the structure on the aspiration catheter to expand or constrict the central clot-receiving passage may comprise torqueing at least a first coil in a first rotational direction to radially open or close the radially distal expandable segment. The first coil may torqued in a first direction to radially expand the radially distal segment of the aspiration catheter and torqued in a second rotational direction to radially constrain the radially distal segment of the aspiration catheter. Torqueing the first coil may comprise rotating an inner member or an outer member attached to a distal end of the first coil, optionally further comprising rotating a second coil attached to a distal end of the first coil.

The methods may result in the clot being extracted substantially intact or in other instances may result in a proximal portion of the clot being extracted substantially intact. Often substantially all clot may be extracted in a first extraction attempt. Often, the extracted clot comprises hard clot.

In still further instances of the methods herein, the scaffold may comprise an element which follows a single path to form a cylindrical or conical envelope. The single path may have anyone or a combination of a closed loop, an open path,

In particular instances, radially expanding the distal portion of the aspiration catheter comprises rotating an inner member attached to the scaffold, wherein the scaffold comprises a cylindrical distal region having a first coil is fixed at its proximal end to a distal end of the catheter body and at its distal end to a distal end of the inner member, wherein rotation of a proximal end of the inner member rotates the distal end of the first coil. The cylindrical distal region of the scaffold may further comprises a rotatable outer member, where the first coil is fixed at its proximal end to a distal end of the catheter body and at its distal end to a distal end of the outer member such that rotation of a proximal end of the outer member rotates the distal end of the first coil.

In yet another aspect of the present invention, an endoluminal prosthesis comprises a scaffold having a plurality of circumferential rings arranged along an axis. The rings comprise struts joined by crowns, typically being patterned from a non-degradable material. The scaffold may be configured to expand from a crimped configuration to an expanded configuration, and at least some of the circumferential rings may be circumferentially separable, typically being joined by circumferentially separable axial links. Thus the scaffold may be configured to circumferentially separate along separation interfaces, where circumferentially separable regions of the circumferential rings and the axial links often comprise a biodegradable polymer and/or adhesive configured to hold said separations regions together during expansion and to thereafter form at least one discontinuity in the circumferential ring and the axial link after expansion of the scaffold in a physiologic environment; As a particular feature, the scaffold is formed one (a single) continuous structure so that it will remain intact along a length of the element after all discontinuities are formed.

In a still further aspect of the present invention, an endoluminal prosthesis comprises a scaffold having a plurality of circumferential rings arranged along an axis. The rings comprise struts joined by crowns and are typically patterned from a non-degradable material. The scaffold being is typically configured to expand from a crimped configuration to an expanded configuration, where at least some of the circumferential rings may be circumferentially separated, often be joined by circumferentially separable axial links, such that the scaffold may be expandable from the crimped configuration to an expanded configuration in a physiologic environment. As a particular feature, the scaffold is formed from one (a single) continuous patterned structure enhancing strength in the expanded configuration to support a body lumen.

In yet an additional aspect of the present invention, an aspiration catheter for removing clot from a blood vessel comprises a catheter body having a proximal end, a distal end, and an aspiration lumen therebetween. A scaffold extends distally from the distal end of the catheter body and typically includes a central clot-receiving passage contiguous with the aspiration lumen of the catheter body. An elastic membrane covering the scaffold establishes a clot aspiration path from a distal end of the scaffold to a proximal end of the lumen in the catheter body so that applying a vacuum to a proximal end of the aspiration lumen can draw clot into the central clot-receiving passage, where at least a distal portion of the scaffold is radially expandable from a delivery configuration to an extraction configuration. As a particular feature, the scaffold comprises two or more circumferential bisected rings with at least one bisected axial connection connecting said bisected rings.

In one example of the present invention, the device comprises an elongated tubular body comprising a distal segment and a proximal segment, wherein the distal segment is expandable from an initial small configuration to a larger configuration and then back down to a final small configuration, wherein the final small configuration is smaller than the larger configuration and may be equal to or larger than the initial small configuration. In the device of the present example the distal end of said distal segment is configured to engage a clot and/or to substantially engage the vessel wall adjacent to a clot, and the elongated tubular body comprises an aspiration lumen, and the device is able to retrieve the clot by applying a vacuum force through the aspiration lumen to the distal end of said distal segment. In an exemplary example, the vacuum force applied is between 10 mmHg and 760 mm Hg, more preferably between 10 mmHg and 380 mm Hg, and more preferably between 10 mm and 200 mm Hg. In a further example of the present example, the elongated tubular body comprises a distal segment, and intermediate or middle segment, and a proximal segment. In another example, the distal segment extends substantially the entire length of the elongated tubular body, and has a length ranging from 1 cm to 50 cm, preferable having a length ranging from 2 cm to 20 cm, more preferably having a length ranging from 3 cm to 15 cm.

In another example, the aspiration lumen diameter of the proximal segment is larger than the aspiration lumen diameter of the distal segment in the contracted configuration but smaller than the aspiration lumen diameter of the distal segment in the expanded configuration.

In an exemplary example, the distal segment small configurations comprise one or more of the following: crimped configuration, collapsed configuration, contracted configuration, unexpanded configuration, unopened configuration, delivery configuration, or other. In another exemplary example, the distal segment larger configuration comprises one or more of the following: deployed configuration, expanded configuration, aspiration configuration, or other.

In an exemplary example, the distal segment is controllably expandable from a smaller configuration to the larger configuration and then controllably contractible to a smaller configuration. In another example, the distal segment is controllably contractible or crimpable to small configuration prior to insertion in a body lumen, then controllably expandable to a larger configuration in a body lumen, and then controllably contractible to a smaller configuration prior to withdrawal of said distal segment from a body lumen.

In an exemplary example, the distal segment is expandable and/or contractible by means of twisting or rotating torque elements attached to each end of a single coil structure in the distal segment, and the torque applied to at least one of the torque elements attached to the single coil structure causes the single coil structure to unwind to expand in diameter, or to wind to contract in diameter.

In another exemplary example, the distal segment is expandable and/or contractible by means of twisting or rotating torque elements attached to two or more coil structures in the distal segment, wherein said two or more coil structures are connected to each other in at least in one location at the distal end of said distal segment, and the proximal ends of the coil structures are connected to said torque elements, and opposing torques applied to at least one of the two or more coil structures cause them to unwind to expand in diameter or to wind to contract in diameter.

In another exemplary example, the distal segment is expandable and/or contractible by means of twisting or rotating torque elements and/or axially compressing or tensioning linear force elements connected to a braided wire structure in the distal segment, wherein the wires of the braid are then forced against each other in order to achieve the expansion or contraction.

In another exemplary example, the distal segment is expandable and/or contractible by means of axially compressing or tensioning linear force elements connected to a removeable and replaceable sleeve over a braided wire structure in the distal segment, wherein the braid is designed to be self-expanding when not constrained by the sheath.

In another exemplary example, the distal segment is expandable and/or contractible by means of axially compressing or tensioning linear force elements connected to a removeable and replaceable sleeve over a structure in the distal segment comprising a slotted tube or sinusoidal ring structure, wherein the slotted tube or sinusoidal wire structure is designed to be self-expanding when not constrained by the sheath.

In another exemplary example, the distal segment is expandable and/or contractible by means of axially compressing or tensioning linear force elements connected to a structure in the distal segment comprising of three or more longitudinally aligned ribs, which when put in compression causes them to bow outwards thereby expanding their profile, and when put in tension causes them to stretch flatter thereby contracting their profile. In a preferred variant of the present example, one or more V links or other means are used to attach the ribs to each other in order to maintain their circumferential alignment.

In an exemplary example, the expansion and contraction of the distal segment is controllable by torque elements and/or linear force elements comprising of one or more of the following: wires, rods, tubes, or other, and the torque elements and/or linear force elements extend substantially along the length of the elongated tubular body. In an exemplary example, the torque elements and/or linear force elements are formed from a metallic, polymeric, or composite material. In the preferred example, at least one torque element and/or linear force element comprises the catheter shafts.

In an exemplary example, the coil, braided wire, sinusoidal ring, or longitudinal rib structure comprises one or more of round wire, tubular wire, flat ribbon, contoured ribbon, or the like. In an exemplary example, the coil, braided wire, sinusoidal ring, or longitudinal rib structure are formed from a metallic material such as stainless steel, cobalt chrome, or other. In an exemplary example, the coil, braided wire, sinusoidal ring, or longitudinal rib structure are formed from a shape memory material such as a nickel-titanium alloy (“NiTi) or the like.

In an exemplary example, a covering sleeve extends over the distal segment of the elongated tubular body, preferably covering substantially the entire length of the distal segment, wherein said covering sleeve accommodates the expansion and contraction of the distal segment while functionally maintaining vacuum pressure integrity in the aspiration lumen of the elongated tubular body. In an exemplary example, the covering sleeve comprises one or more of the following: spray coated sleeve, dip coated sleeve, elastic sleeve, radially expandable elastic sleeve, polymeric sleeve, foldable sleeve, silicone based material sleeve, polyurethane based sleeve, and other. The sleeve is preferably attached to the distal segment in one or more locations but can alternatively be press fit onto the distal segment without attachment.

In an exemplary example, the covering sleeve only partially covers the distal segment of the elongated tubular body, such that a distal portion of the distal segment is uncovered and the expansion/contraction structure is able to directly engage the clot. An associated method of use is to advance the device until the portion of the distal segment without the covering sleeve is within the clot, such that expansion of said portion of the distal segment causes the uncovered structure to directly engage the clot, thereby aiding with breaking up the clot for improved aspiration or snaring it for withdrawal from the anatomy. In this method the distal segment may also be manipulated linearly or rotationally as part of the procedure to improve such engagement and effects, and the distal uncovered portion of the expansion structure may furthermore incorporate features to improve cutting of or gripping the clot such as sharper edges, metallic protrusions, fins, hook elements, slots in the coil ribbon, and other.

In an exemplary example, the proximal and/or intermediate segments of the elongated tubular body are comprised of a polymeric material, which may or may not contain a polymeric or metallic coil or braid within or adjacent to the polymeric material.

In an exemplary example, the distal segment is expandable from a contracted configuration to an expanded configuration, wherein the outer diameter or the aspiration lumen diameter of the distal segment in said expanded configuration is substantially the same as a non-occluded lumen diameter of the vessel adjacent to said expanded distal segment. In another example, the distal segment is controllably expandable from a contracted configuration to an expanded configuration wherein the outer diameter or the aspiration lumen diameter of the distal segment in the expanded configuration ranges from 0.5 times the non-occluded vessel lumen diameter to 1.2 times the non-occluded vessel lumen diameter, preferably the expanded configuration ranges from 0.75 times the non-occluded vessel lumen diameter to 1.2 times the non-occluded vessel lumen diameter, more preferably is substantially the same diameter of the non-occluded vessel lumen.

In another example, the present invention comprises an aspiration catheter having a distal segment configured to expand to a range of diameters ranging from 0.5 mm outer diameter in the fully collapsed state to 5.0 mm outer diameter in the fully expanded state. The device is advanced in a patient body with the distal segment in a small collapsed state to navigate the tortuous vasculature until reaching the occluded vessel and/or clot. Once the distal end of the distal segment or tip is positioned adjacent to or contacting the clot or thrombus, the distal segment of the device is expanded to a larger diameter to increase its tip area and the vacuum effectiveness. In an exemplary example, the expandable distal segment is expanded until it substantially contacts the vessel wall, in order to enhance clot separation from the vessel wall and clot removal. Advantages of expanding the distal segment or distal end of the catheter to substantially the vessel size include one or more of the following: separating the clot from the vessel wall, ease of retrieving the clot with small to modest suction force, retrieving the clot substantially intact or with less fragments, removal of the clot substantially from first attempt. The distal segment may be expanded larger than the vessel in order to further enhance clot separation from the vessel wall and clot removal. Once clot retrieval into the catheter is accomplished, the device is then reduced in size again to aid withdrawal of the aspiration system from the anatomy and minimize vascular trauma.

In another example, the device provides improved distal access in tortuous anatomy, greater revascularization success rate, shorter procedure time due to improved first pass revascularization rates and immediate clot retrieval, and reduced risk of distal emboli, all with a single device treatment approach. Furthermore, in many cases a clot may be able to be removed using a low to medium vacuum pressure, potentially further reducing vessel trauma.

Thus, in accordance with at least some of the principles of the present invention as set forth above, an aspiration catheter for removing clot from a blood vessel comprises a catheter body having a proximal end, a distal end, and an aspiration lumen therebetween, a radially distal expandable segment having a central clot-receiving passage extends distally from the distal end of the catheter body, and a means is integrated with the catheter body for expanding and/or constricting the radially distal expandable segment between a radially expanded configuration and a radially contracted configuration. The aspiration lumen of the catheter body and the central clot-receiving passage of the radially distal expandable segment are contiguous so that applying a vacuum to a proximal end of the aspiration lumen can draw clot into the central clot-receiving passage.

The radially distal expandable segment may be self-expanding, for example where the expanding means comprises a sheath configured to constrain the radially distal expandable segment in a radially constrained configuration, where retraction of the sheath allows the radially distal expandable segment to radially expand.

Alternatively or additionally, the expanding means may be integrated with the catheter body, for example comprising (1) at least a first coil which is configured to be torqued in at least one rotational direction to radially open or close the radially distal expandable segment and (2) an elastic sleeve which covers the first coil to enclose the central clot-receiving passage to create a continuous vacuum path from the aspiration lumen to a distal end of the radially distal expandable segment. The first coil may be configured to be torqued in both rotational directions to radially open and radially close the radially distal expandable segment, respectively. In a first instance, torqueing may be accomplished by a rotatable inner member, wherein the first coil is fixed at its proximal end to a distal end of the catheter body and at its distal end to a distal end of the inner member, wherein rotation of a proximal end of the inner member rotates the distal end of the first coil. In a second instances, torqueing may be accomplished by a second coil rotatably and coaxially mounted within the at least first coil, wherein the at least one coil is fixed at its proximal end to a distal end of the catheter body and at its distal end to a distal end of the second coil, wherein the first and second coils are wound in opposite helical directions so that rotation of a proximal end of the second coil in a first direction causes both the first and second coils to radially expand.

Further in accordance with at least some of the principles of the present invention as set forth above, a method for extracting clot from a blood vessel comprises positioning a radially expandable distal segment of an aspiration catheter in a blood vessel proximal to the clot. The radially distal segment of the aspiration catheter is radially expanded in the blood vessel to form an enlarged central clot-receiving passage contiguous with an aspiration lumen in the aspiration catheter. A vacuum is applied to a proximal portion of the aspiration lumen to draw clot from the blood vessel into the enlarged central clot-receiving passage. The radially expandable distal segment of the aspiration catheter is radially constricted in the blood vessel to close the central clot-receiving passage, and at least one of the radially expanding step and the radially constricting step comprises actuating structure on the aspiration catheter to open or close the central clot-receiving passage.

These methods may comprise any of the features of the present invention described previously with respect to the apparatus. For example, the radially expandable distal segment may be self-expanding and radially expanding the radially expandable distal segment may comprise releasing the radially distal expandable segment from a constraining sheath. Alternatively, radially expanding/contracting the radially expandable distal segment may comprise actuating a structure on the aspiration catheter to radially expand/contract the radially expandable distal segment. For example, actuating the structure on the aspiration catheter to expand or constrict the central clot-receiving passage may comprise torqueing at least a first coil in a first rotational direction to radially open or close the radially distal expandable segment. Optionally, the first coil may be torqued in a first direction to radially expand the radially distal segment of the aspiration catheter comprises and further torqued in a second rotational direction to radially constrain the radially distal segment of the aspiration catheter. Torqueing the first coil comprises may comprise rotating an inner member attached to a distal end of the first coil. Alternatively, torqueing the first coil may comprise rotating a second coil attached to a distal end of the first coil.

In some instances, a self-expanding radially expandable distal segment may be expanded by release from a constraining sheath and constricted by actuating a structure on the aspiration catheter to radially contract the radially expandable distal segment.

Still further in accordance with at least some of the principles of the present invention as set forth above, a catheter for resecting and aspirating clot from a blood vessel comprises a catheter body having a proximal end, a distal end, and an aspiration lumen therebetween. A radially expandable scaffold having a central clot-receiving passage extends distally from the distal end of the catheter body, and at least a distal portion of the radially expandable scaffold is configured be disrupt a region of clot when radially expanded therein. The central clot-receiving passage is configured to pass disrupted clot into the aspiration lumen when a vacuum is applied to a proximal end of the aspiration lumen.

The catheter for resecting and aspirating clot of the present invention may further comprise an elastic sleeve that covers at least a proximal portion of the radially expandable scaffold, typically leaving a distal resection portion uncovered. The elastic sleeve is typically configured to cover central clot-receiving passage to create a continuous vacuum path through the central clot-receiving passage and into a distal end of the aspiration lumen, thus allowing resected clot to be aspirated directly from the central clot-receiving passage, through the aspiration lumen in the aspiration catheter, and to an external vacuum collection receptacle.

The aspiration catheter body may further comprise means integrated with the catheter body for expanding and/or constricting the radially expandable scaffold between a radially expanded configuration and a radially contracted configuration. For example, the means integrated with the catheter body for expanding and/or constricting the radially distal expandable segment may comprise at least a first coil which is configured to be torqued in at least one rotational direction to radially open or close the radially distal expandable segment, wherein the first coil is typically configured to be torqued in both rotational directions to radially open and close the radially distal expandable segment. For example, the first coil may be fixed at its proximal end to a distal end of the catheter body and at its distal end to a distal end of the inner member, wherein rotation of a proximal end of the inner member rotates the distal end of the first coil. Alternatively, a second coil may be rotatably and coaxially mounted within the at least first coil, where the at least one coil is fixed at its proximal end to a distal end of the catheter body and at its distal end to a distal end of the second coil. The first and second coils may be wound in opposite helical directions so that rotation of a proximal end of the second coil in a first direction causes both the first and second coils to radially expand.

In other instances, the radially expandable scaffold may self-expanding, and the catheter may further comprise a sheath configured to constrain the radially expandable scaffold in a radially constrained configuration, wherein retraction of the sheath allows the radially distal expandable segment to radially expand. The radially expandable scaffold may comprise closed cells, serpentine rings, axial struts, or may have any of a variety of other scaffold constructions commonly used in constructing vascular prostheses.

In yet further accordance with at least some of the principles of the present invention as set forth above, a method for disrupting and extracting clot from a blood vessel comprises positioning a radially expandable scaffold or expandable coil at a distal end of an aspiration catheter within a region of clot in a blood vessel. The radially expandable scaffold or expandable coil is radially expanded within the region of clot and disrupted clot is collected within a central clot-receiving passage of the scaffold which is contiguous with an aspiration lumen in the aspiration catheter. By applying a vacuum to a proximal portion of the aspiration lumen, disrupted clot may be extracted from the central clot-receiving passage into the lumen.

The radially distal expandable segment may be self-expanding and expanding the radially expandable distal segment of the aspiration catheter may comprise releasing the radially distal expandable segment from a constraining sheath. Alternatively, radially expanding the radially expandable distal segment of the aspiration catheter may comprise actuating structure on the aspiration catheter to radially expand the radially expandable distal segment. For example, actuating structure on the aspiration catheter to expand or constrict the central clot-receiving passage may comprise torqueing at least a first coil in a first rotational direction to radially open or close the radially distal expandable segment. The first coil may be torqued in a first direction to radially expand the radially distal segment of the aspiration catheter and torqued in a second rotational direction to radially constrain the radially distal segment of the aspiration catheter. Torqueing the first coil may comprise rotating an inner member attached to a distal end of the first coil. Alternatively, torqueing the first coil may comprise rotating a second coil attached to a distal end of the first coil.

In an exemplary example, the expandable distal segment comprises a self-expanding structure which is constrained in the smaller configuration and then released and/or allowed to self-expand to the larger configuration.

In an exemplary example, the expandable distal segment comprises a self-expanding structure which is constrained by an outer sheath partially or fully covering the expandable distal segment, and the outer sheath is moved proximally relative to the expandable distal segment and/or the expandable distal segment is moved distally relative to the outer sheath, thereby releasing the constraint and allowing the self-expanding structure to self-expand.

In an exemplary example, the expandable distal segment comprises a self-expanding structure which is constrained by a sheath (or cap) at its distal end partially or fully covering the expandable distal segment, and the sheath or cap is moved distally relative to the expandable distal segment and/or the sheath or cap is moved proximally relative to the expandable distal segment and/or the expandable distal segment is moved proximally relative to the sheath or cap, thereby releasing the constraint and allowing the self-expanding structure to self-expand. In one example, the sheath (or cap) are controlled by a wire or tube slidably movable inner to the expandable structure.

In an exemplary example, the expandable distal segment comprises a self-expanding structure comprising struts, tines, hooks, or other means by which the expandable distal segment is constrained from the inside by a wire or inner elongated tubular body inside the outer elongated tubular body, and the wire or inner elongated tubular body is moved proximally within the outer elongated tubular body to release the constraint and allowing the self-expanding structure to self-expand.

In an exemplary example, the expandable distal segment comprises a self-expanding structure comprising holes or loops within its structure and the expandable distal segment is constrained from the inside by wires or inner elongated tubular body inside the outer elongated tubular body shaped to engage such holes or loops, and the wire or inner elongated tubular body is moved proximally within the outer elongated tubular body to release the constraint and allowing the self-expanding structure to self-expand.

In an exemplary example, the expandable distal segment comprises a self-expanding structure, which is covered by a constraining ring over the distal portion of the expandable distal segment, and the ring is moved proximally to partially or fully uncover the distal portion of the expandable distal segment, thereby allowing the self-expanding structure to self-expand.

In an exemplary example, the expandable distal segment comprises a self-expanding structure which naturally remains in the smaller configuration until exposed to heat such as about 37 degrees Celsius and/or moisture such as body moisture, which allows it to self-expand to the larger configuration.

In an exemplary example, the expandable distal segment comprises a self-expanding structure which naturally remains in the smaller configuration until charged with an electric current, which allows it to self-expand to the larger configuration.

In an exemplary example, the expandable distal segment comprises a self-expanding structure comprising linear elements or axial tines which are in a neutral state when in the larger configuration, and yield elastically when bent or compressed into the smaller configuration, from which they seek to elastically expand back to the larger configuration.

In an exemplary example, the expandable distal segment comprises a self-expanding structure comprising one or more sinusoidal rings which are in a neutral state when in the larger configuration, and yield elastically when compressed into the smaller configuration, from which they seek to elastically expand back to the larger configuration.

In an exemplary example, the expandable distal segment comprises a self-expanding structure comprising linear elements or axial tines and one or more sinusoidal rings which are in a neutral state when in the larger configuration, and yield elastically when compressed into the smaller configuration, from which they seek to elastically expand back to the larger configuration.

In an exemplary example, the expandable distal segment comprises an expandable structure which can be mechanically manipulated from the smaller configuration to the larger configuration by means of one or more of pushing, pulling, or torqueing wires, rods or tubes incorporated into the device, by means of pneumatic or hydraulic pressure, or by other means.

In an exemplary example, the expandable distal segment comprises a sleeve covering part or all of the expandable distal segment separate from the constraining means (or constraining sheath). This sleeve allows one or more of the following: hold a vacuum during aspiration, hinder back flow of blood into the aspiration device, maximize the pressure gradient to aspirate the clot.

In an exemplary example, the expandable distal segment is expanded to a larger configuration substantially apposing the vessel wall and sufficiently holding a vacuum to aspirate a clot or hinder back flow of blood into the aspiration catheter. In this example, the vessel wall behaves like a sleeve to offer the holding of vacuum, prevent blood from substantially getting into the aspiration catheter, and/or maximize the pressure gradient to aspirate a clot. In one example, substantially all of the expandable distal segment apposes the vessel wall.

In an exemplary example, the expandable distal segment of any of the examples, wherein it expands from a crimped configuration to an expanded configuration, said expandable configuration being larger than the constraining means configuration and smaller than 1.1 times the configuration of the vessel adjacent to the expandable distal end. In a preferred example, the expandable configuration of the distal end is expanded to about the inner vessel configuration adjacent to the expandable distal end. In another example, the configuration is the diameter of the expandable segment, the vessel, and/or the sheath.

In an exemplary example, the expandable distal segment of any of these examples, comprises one or more circumferential rings, wherein said one or more circumferential rings being expandable from a crimped configuration to an expanded configuration. In one example, the circumferential rings comprise struts joined by crowns. In another example, the circumferential rings comprise two or more rings wherein adjacent rings are joined by one or more links. In another example, the circumferential rings comprise two or more rings wherein adjacent rings are joined by one or more axial links. In another example, the expandable distal segment comprises an expandable funnel-like structure, typically comprising three or more axial elements shiftable between a cylindrical configuration where the elements are axially aligned and an expanded configuration where they diverge outwardly in a distal direction. In another example, the expandable distal segment is an umbrella-like structure comprising two or more axial struts expandable from a crimped configuration to an expanded configuration and wherein one or more expandable rings joins said two or more axial struts. In yet another example, the expandable distal segment comprises one or more circumferential rings wherein the rings expand circumferentially from a crimped configuration to an expanded configuration.

In yet another example, the expandable distal segment extends proximally a length ranging from 1 mm to 150 cm, preferably ranging from 2 mm to 20 cm, more preferably ranging from 3 mm to 10 cm, and most preferably ranging from 3 to 10 mm.

In yet another example, the expandable distal segment is deployed to an expanded configuration from a crimped configuration to aspirate a clot distal to the expanded segment, and then said expanded distal segment is optionally collapsed to a smaller configuration by prior to repositioning the device within the anatomy for further aspiration procedures or withdrawing the aspiration catheter system. Means for collapsing the expandable distal segment include pulling or pushing said expanded segment into a sheath, pulling a drawstring-type thread or wire, rotating torque members to wind a coil to a tighter diameter, and other means described elsewhere herein.

In another example, the distal expandable segment has a flexibility and bendability sufficient to allow said segment to reach one or more of a blocked blood vessel in the brain, a location adjacent to a blocked blood vessel in the brain, and a location proximal to a blocked blood vessel in the brain, often a middle cerebral artery.

In another example, the distal expandable segment is configured to have flexibility sufficient to allow the distal expandable segment to reach one or more of a blocked artery in the brain, adjacent to a blocked artery in the brain, proximal to a blocked artery in the brain, middle cerebral artery.

In another example, the distal expandable segment is configured to have flexibility in all axis wherein said flexibility in said two or more axis is sufficient to allow said segment to reach one or more of a blocked artery in the brain, adjacent to a blocked artery in the brain, proximal to a blocked artery in the brain, middle cerebral artery.

In yet another example, the expandable distal segment is substantially tubular in the crimped configuration.

In yet another example, the expandable distal segment is substantially tubular in the crimped configuration and is expandable into a funnel shaped structure comprising one or more expandable elements and a sleeve covering said expandable elements.

In yet another example, the expandable distal segment is substantially tubular in the crimped configuration and is expandable into a funnel shaped structure comprising an expandable elements and a sleeve covering said expandable elements, wherein said funnel has an angle ranging from 100 degrees to 150 degrees from the delivery system, or from 10 degrees to 80 degrees from the delivery system, wherein said funnel angle is configured to inhibit collapse of said funnel when a vacuum force ranging from 50 mmHg to 760 mmHg is applied proximally to said funnel. In one example, the funnel expands distally towards the clot. In another example, the funnel expands proximally away from the clot.

In yet another example, the expandable distal segment is substantially tubular in the crimped configuration and is expandable into a funnel shaped structure comprising one or more expandable elements and a sleeve covering said expandable elements and wherein the funnel comprises an end segment configured to be substantially parallel to the vessel wall.

In a preferred example, the expandable distal segment is expandable to a configuration ranging from 0.7 times the adjacent vessel inner configuration to 1.1 times the adjacent inner vessel inner configuration to allow sufficient vacuum to remove a clot, preferably expandable to about the adjacent inner vessel configuration to allow sufficient vacuum to remove a clot.

In another example, the distal expandable segment is formed from metal, metal alloy, or polymeric material, wherein said expandable material comprises shape memory alloy or shape memory polymeric material.

Note: the terms flexibility and stiffness are commonly used when describing performance of medical devices, especially those like the present invention which need to track through blood vessels to reach the site of treatment. Stiffness is most commonly quantitatively characterized by a Three Point Bend Test, in which a portion of the scaffold, shaft, or other device component is supported on its edges by a rigid fixture while an anvil is pressed against the center of the component between the supports to force it into a curve. A load cell or other force measuring tool attached to the anvil measures the force required to bend the test unit. The stiffness of the test unit can therefore be characterized in terms of Force per Distance, such as Newtons per millimeter. The stiffness of the device is sometimes referred to simply in terms of force, i.e. 0.6 N, when the test setup is the same for all samples in the test group such that the “per distance” aspect is common to all. As an example of a particular Three Point Bend Test setup, a product designed to be tracked in a tortuosity with a mean radius of curvature of 6.5 mm uses a three point bend test fixture with the side supports 13 mm apart, and an anvil compression distance of 2 mm in order to fully load the test samples while keeping the bending substantially in the elastic range. In such an example, a peak load of 0.6 N would correspond to an average stiffness of 0.3 N/mm. Flexibility is the qualitative inverse of stiffness—a device which is more stiff than whatever it is being compared to is less flexible, and vice versa.

Other methods commonly used to assess the acute delivery performance of medical devices include Track and Push tests. A Track Test involves clamping the test device to a fixture connected to a load cell, which advances the catheter through a tortuosity while measuring the force to do so with the load cell. In this case the Force per Distance output data tends to form a series of sinusoids with peaks of increasing elevation, where each rise in data corresponds to the force required to advance the device through a particular curve in the anatomy. Typically the data in analyzed in terms of peak force—the greatest amount of force required to advance the device through any point in the fixture. Data can also be analyzed in terms of average force over the whole distance, average force for a section (such as around a particular curve), or even distance advanced until a certain force ceiling is hit. A Push test uses a generally similar test setup, except a second load cell is anchored somewhere in the tortuosity, and the test device advanced until its tip is in contact with the second load cell. The test device is then further advanced thereby putting the device in compression between the load cells, and the efficiency of force transmission from the proximal to the distal load cell determined. For example if the proximal load cell reads 1.0 N applied force and the distal load cell reads 0.3 N at the catheter tip, push transmission is 30%.

The inventions claimed herein are further set forth and described in the following numbered clauses:

Clause 1. An aspiration catheter for removing clot from a blood vessel, said catheter comprising: a catheter body having a proximal end, a distal end, and an aspiration lumen therebetween; a radially distal expandable segment having a central clot-receiving passage extending distally from the distal end of the catheter body; and means integrated with the catheter body for expanding and/or constricting the radially distal expandable segment between a radially expanded configuration and a radially contracted configuration; wherein the aspiration lumen of the catheter body and the central clot-receiving passage of the radially expandable separator scaffold are contiguous so that applying a vacuum to a proximal end of the aspiration lumen can draw clot into the central clot-receiving passage.

Clause 2. The aspiration catheter of clause 1, wherein the radially distal expandable segment is self-expanding.

Clause 3. The aspiration catheter of clause 2, further comprising a sheath configured to constrain the radially distal expandable segment in a radially constrained configuration, wherein retraction of the sheath allows the radially distal expandable segment to radially expand.

Clause 4. The aspiration catheter of clause 1, wherein the means integrated with the catheter body for expanding and/or constricting the radially distal expandable segment comprises (1) at least a first coil which is configured to be torqued in at least one rotational direction to radially open or close the radially distal expandable segment and (2) an elastic sleeve which covers the first coil to enclose the central clot-receiving passage to create a continuous vacuum path from the aspiration lumen to a distal end of the radially distal expandable segment.

Clause 5. The aspiration catheter of clause 4, wherein the first coil is configured to be torqued in both rotational directions to radially open and close the radially distal expandable segment.

Clause 6. The aspiration catheter of clause 4 or 5, further comprising a rotatable inner member, wherein the first coil is fixed at its proximal end to a distal end of the catheter body and at its distal end to a distal end of the inner member, wherein rotation of a proximal end of the inner member rotates the distal end of the first coil.

Clause 7. The aspiration catheter of clause 4 or 5, further comprising a second coil rotatably and coaxially mounted within the at least first coil, wherein the at least one coil is fixed at its proximal end to a distal end of the catheter body and at its distal end to a distal end of the second coil, wherein the first and second coils are wound in opposite helical directions so that rotation of a proximal end of the second coil in a first direction causes both the first and second coils to radially expand.

Clause 8. A method for extracting clot from a blood vessel, said method comprising: positioning a radially expandable distal segment of an aspiration catheter in a blood vessel proximal to the clot; radially expanding the radially distal segment of the aspiration catheter in the blood vessel to form an enlarged central clot-receiving passage contiguous with an aspiration lumen in the aspiration catheter; applying a vacuum to a proximal portion of the aspiration lumen to draw clot from the blood vessel into the enlarged central clot-receiving passage; and radially constricting the radially distal segment of the aspiration catheter in the blood vessel to close central clot-receiving passage; wherein at least one of radially expanding and radially constricting the distal segment of the aspiration catheter comprises actuating structure on the aspiration catheter to open or close the central clot-receiving passage.

Clause 9. The method of clause 8, wherein the radially distal expandable segment is self-expanding and radially expanding the radially distal segment of the aspiration catheter comprises releasing the radially distal expandable segment from a constraining sheath.

Clause 10. The method of clause 8, wherein radially expanding the radially expandable distal segment of the aspiration catheter comprises actuating the structure on the aspiration catheter to radially expand the radially expandable distal segment.

Clause 11. The method of clause 8, wherein actuating structure on the aspiration catheter to expand or constrict the central clot-receiving passage comprises torqueing at least a first coil in a first rotational direction to radially open or close the radially distal expandable segment.

Clause 12. The method of clause 11, wherein the first coil is torqued in a first direction to radially expand the radially distal segment of the aspiration catheter comprises and torqued in a second rotational direction to radially constrain the radially distal segment of the aspiration catheter.

Clause 13. The method of clause 11 or 12, wherein torqueing the first coil comprises rotating an inner member attached to a distal end of the first coil.

Clause 14. The method of clause 11 or 12, wherein torqueing the first coil comprises rotating a second coil attached to a distal end of the first coil.

Clause 15. A catheter for resecting and aspirating clot from a blood vessel, said catheter comprising: a catheter body having a proximal end, a distal end, and an aspiration lumen therebetween; and a radially expandable scaffold having a central clot-receiving passage extending distally from the distal end of the catheter body; wherein at least a distal portion of the radially expandable scaffold is configured be disrupt a region of clot when radially expanded therein and wherein the central clot-receiving passage is configured to pass disrupted clot into the aspiration lumen when a vacuum is applied to a proximal end of the aspiration lumen.

Clause 16. The aspiration catheter of clause 15, further comprising an elastic sleeve that covers a proximal portion of the radially expandable scaffold, leaving a distal resection portion uncovered, wherein the elastic sleeve is configured to create a continuous vacuum path through the central clot-receiving passage and into a distal end of the aspiration lumen.

Clause 17. The aspiration catheter of clause 15 or 16, further comprising means integrated with the catheter body for expanding and/or constricting the radially expandable scaffold between a radially expanded configuration and a radially contracted configuration.

Clause 18. The aspiration catheter of clause 17, wherein the means integrated with the catheter body for expanding and/or constricting the radially distal expandable segment comprises at least a first coil which is configured to be torqued in at least one rotational direction to radially open or close the radially distal expandable segment.

Clause 19. The aspiration catheter of clause 18, wherein the first coil is configured to be torqued in both rotational directions to radially open and close the radially distal expandable segment.

Clause 20. The aspiration catheter of clause 19, further comprising a rotatable inner member, wherein the first coil is fixed at its proximal end to a distal end of the catheter body and at its distal end to a distal end of the inner member, wherein rotation of a proximal end of the inner member rotates the distal end of the first coil.

Clause 21. The aspiration catheter of clause 19, further comprising a second coil rotatably and coaxially mounted within the at least first coil, wherein the at least one coil is fixed at its proximal end to a distal end of the catheter body and at its distal end to a distal end of the second coil, wherein the first and second coils are wound in opposite helical directions so that rotation of a proximal end of the second coil in a first direction causes both the first and second coils to radially expand.

Clause 22. The aspiration catheter of clause 15, wherein the radially expandable scaffold is self-expanding, said catheter further comprising a sheath configured to constrain the radially expandable scaffold in a radially constrained configuration, wherein retraction of the sheath allows the radially distal expandable segment to radially expand.

Clause 23. The aspiration catheter of clause 22, wherein the radially expandable scaffold comprises closed cells.

Clause 24. The aspiration catheter of clause 22, wherein the radially expandable scaffold comprises serpentine rings.

Clause 25. The aspiration catheter of clause 22, wherein the radially expandable scaffold comprises axial struts.

Clause 26. A method for disrupting and extracting clot from a blood vessel, said method comprising: positioning a radially expandable scaffold at a distal end of an aspiration catheter within a region of clot in a blood vessel; radially expanding the radially expandable scaffold within the region of clot and collecting disrupted clot within a central clot-receiving passage of the scaffold which is contiguous with an aspiration lumen in the aspiration catheter; and applying a vacuum to a proximal portion of the aspiration lumen to draw disrupted clot from the central clot-receiving passage into the lumen.

Clause 27. The method of clause 26, wherein the radially distal expandable segment is self-expanding and radially expanding the radially distal segment of the aspiration catheter comprises releasing the radially distal expandable segment from a constraining sheath.

Clause 28. The method of clause 26, wherein radially expanding the radially expandable distal segment of the aspiration catheter comprises actuating the structure on the aspiration catheter to radially expand the radially expandable distal segment.

Clause 29. The method of clause 28, wherein actuating structure on the aspiration catheter to expand or constrict the central clot-receiving passage comprises torqueing at least a first coil in a first rotational direction to radially open or close the radially distal expandable segment.

Clause 30. The method of clause 29, wherein the first coil is torqued in a first direction to radially expand the radially distal segment of the aspiration catheter comprises and torqued in a second rotational direction to radially constrain the radially distal segment of the aspiration catheter.

Clause 31. The method of clause 29 or 30, wherein torqueing the first coil comprises rotating an inner member attached to a distal end of the first coil.

Clause 32. The method of clause 29 or 30, wherein torqueing the first coil comprises rotating a second coil attached to a distal end of the first coil.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative examples, in which the principles of the invention are utilized, and the accompanying drawings of which:

FIGS. 1A and 1B show an aspiration catheter constructed in accordance with the principles of the present invention having a distal scaffold portion in radially collapsed and expanded configurations, respectively.

FIGS. 2A and 2B show detailed views of a helical scaffold as used in the aspiration catheters of FIGS. 1A and 1B.

FIG. 3 shows a close up of one example of the catheter outer shaft.

FIG. 4 shows a close up of one example of the device handle comprising a fixed handle body 40 and rotating handle knob 41

FIGS. 5A-5G depict an example of the device of the present invention in use.

FIG. 6A shows a helical scaffold comprising a ribbon with a counter-clockwise wind as viewed from proximal to distal, such that the inner torque member would be rotated counter-clockwise to collapse the coil and clockwise to expand it.

FIG. 6B shows a ribbon similar to that of FIG. 6A with a clockwise wind as viewed from proximal to distal, such that the inner torque member would be rotated clockwise to collapse the coil and counter-clockwise to expand it.

FIG. 7 shows an example of a coil with a variable ribbon width along its length.

FIG. 8 shows an example of a double helix coil.

FIG. 9 shows an example of a triple helix coil with the first ribbon and second ribbon having slots cut in the core of the ribbon.

FIGS. 10A and 10B show a collapsed coil and expanded coil, respectively, featuring laser cut notches and bumps.

FIG. 11A shows an example of a coil with a sinusoidal ring ribbon comprising struts joined by crowns.

FIG. 11B shows a side cross section of the distal end of the device of FIG. 11A in an expanded configuration showing the sinusoidal ring ribbon along with an inner torque member and an intermediate outer segment.

FIG. 11C shows a side cross section of the distal end of the device FIG. 11A in a collapsed configuration including the presence of a constraining sleeve.

FIGS. 12 and 13 illustrate a preferred example of the present invention, in which the distal part of the inner torque member has been replaced with a second inner coil wound in direction opposite to that of the outer coil in expanded and collapsed configurations, respectively.

FIG. 14 shows another example of the present invention in which tubular attachments with slots are employed to maintain a constant spacing of the coil ribbon through diametric changes.

FIG. 15 shows an example of a self-expanding conical scaffold with a distal end and a proximal end, comprising a plurality of struts 152 radiating in a distal direction from a common circular base 153.

FIG. 16 shows a self-expanding scaffold having conical and cylindrical segments in which the struts have bends allowing the expanded scaffold to better conform to the vessel in the expanded state for superior vacuum sealing.

FIG. 17 shows a variant of the scaffold of FIG. 16 in which the rounded tips of the struts have flat portions on the leading edge to further reduce vessel trauma and/or better distribute loads against the vacuum-resistant membrane.

FIG. 18 shows another self-expanding scaffold having conical and cylindrical segments in which two or more struts are connected by arcs to an adjacent strut thereby forming loops.

FIG. 19 shows a self-expanding conical scaffold in which the proximal ends of the struts are connected by arc forming a sinusoidal ring or serpentine structure.

FIG. 20 shows a variant of the self-expanding conical scaffold of FIG. 19 in which the struts have bends near the crown tips allowing the expanded scaffold to better conform to the vessel in the expanded state for superior vacuum sealing.

FIG. 21 shows a self-expanding scaffold comprising a plurality of struts connected by arcs at both ends to form a sinusoidal ring structure where a proximal end is attached to a scaffold base by struts.

FIG. 22 shows a self-expanding conical scaffold similar to FIG. 21 in which the links include spring elements to increase flexibility of the self-expanding scaffold as a whole.

FIG. 23 shows an example of conical scaffold formed with a tapered serpentine body attached to a base ring which expands radially outwardly at an angle in a distal direction.

FIG. 24 shows a conical scaffold having a proximal region oriented at a first relative to an axis and a distal region oriented at a second angle relative to the axis, where the first angle is greater than the second angle.

FIG. 25 shows a conical scaffold with radially inward-pointing tips.

FIG. 26 shows a variant of the conical scaffold of FIG. 25 in which the scaffold has more gradually curved inward-pointing tips.

FIG. 27 shows a self-expanding scaffold mounted with the radially converging apical end of the scaffold oriented in a distal direction and the radially diverging end of the scaffold oriented in a proximal direction.

FIG. 28 shows a preferred aspiration catheter of the present invention comprising a self-expanding scaffold attached to an inner elongated tubular body translatably received in an outer elongated tubular body.

FIG. 29 shows an aspiration catheter in which the self-expanding scaffold is radially restrained by a distal cap attached to a removable inner elongated tubular body.

FIG. 30 shows an aspiration catheter having a self-expanding scaffold attached to a distal end of an outer elongated tubular body and constrained by a wire, filament, or ribbon wrapped around the at least distal end of the self-expanding.

FIG. 31 shows an aspiration catheter having a self-expanding scaffold attached to the distal end of the outer elongated tubular body and held in a constrained state by a frangible material.

FIGS. 32A and 32B show an aspiration catheter in which a self-expanding scaffold is attached to a distal end of an elongated tubular body 321 and is constrained state by a drawstring filament.

FIGS. 33A and 33B show an aspiration catheter in which a self-expanding scaffold includes struts 331 of different lengths.

FIG. 34 shows an aspiration catheter in which a self-expanding scaffold is attached to the distal end of an elongated tubular body and constrained state by a ring.

FIGS. 35A and 35B shows an aspiration catheter in which a self-expanding scaffold is compressed and folded into a fixed-diameter aspiration lumen (FIG. 35A) and expanded upon distal advance (FIG. 35B).

FIGS. 36A and 36B are side and end views of an aspiration catheter in which the scaffold may constrained in an aspiration lumen.

FIG. 37 shows an aspiration catheter having a scaffold comprising sinusoidal rings made from a swellable polymer.

FIG. 38 shows a flexible junction design in which a distal expanding segment is coupled to a base which may be attached to a catheter shaft by a coil or pigtail structure.

FIG. 39 shows a distal expanding structure connected to a distal end of a catheter shaft by flexible links.

FIG. 40 shows an expanding structure connected to an adjacent catheter by ball-and-socket type joints.

FIG. 41 shows a distal expanding structure which is disconnected from an adjacent catheter shaft.

FIG. 42 shows a distal expandable segment comprising an ovoidal braided structure which flares outwards from the catheter shaft.

FIG. 43 shows a distal expandable segment comprising a flared braided structure extending from an outer catheter shaft.

FIGS. 44A and 44B are side and end views, respectively, showing a distal expandable segment comprising a longitudinal ribbed structure.

FIG. 45 shows a distal expandable segment comprising multiple rings connected on opposite sides to spines.

FIG. 46 illustrates a distal expandable segment having rings and a single spine covered by a tubular structure with cuts.

FIG. 47 shows an aspiration catheter in which a distal expandable segment comprises a plastically deformable scaffold mounted on an end of an outer elongated tubular body where a balloon catheter may be inflated to expand the scaffold.

FIG. 48 shows another example of the present invention in which the distal expandable segment is constructed from a coiled polymer tubing 480 wherein the coil loops are bonded together.

FIG. 49 shows a distal expanding segment comprising a coil attached to a catheter shaft in which a vacuum resistant membrane extends from an end of the catheter shaft to a point substantially proximal to the distal end of the coil.

FIG. 50 shows a distal expanding segment comprising a self-expanding scaffold attached to a catheter shaft in which a vacuum resistant membrane 502 extends from an end of the catheter shaft to a point substantially proximal to the distal end of a self-expanding scaffold.

FIGS. 51A-51C show a distal expandable scaffold consisting of a single undulating element. The pattern in a flattened (rolled out) state is shown in FIG. 51A and in a rolled configuration in FIGS. 51B (collapsed) and 51C (expanded).

FIGS. 52A and 52B show another example of a distal expandable scaffold consisting of a single undulating element where the scaffold comprises multiple continuous undulating elements which are maintained in position by tab-and-slot joints.

FIG. 53 shows another example of a distal expandable scaffold consisting of a single undulating element attached to an intermediate segment of an aspiration catheter and covered with the vacuum resistant membrane.

### DETAILED DESCRIPTION OF THE INVENTION

### Example 1: Reversibly Expanding Coil

The novel features of the invention are set forth with particularity in the appended claims. A better understanding of the features and advantages of the present invention will be obtained by reference to the following detailed description that sets forth illustrative examples, in which the principles of the invention are utilized, and the accompanying drawings of which:

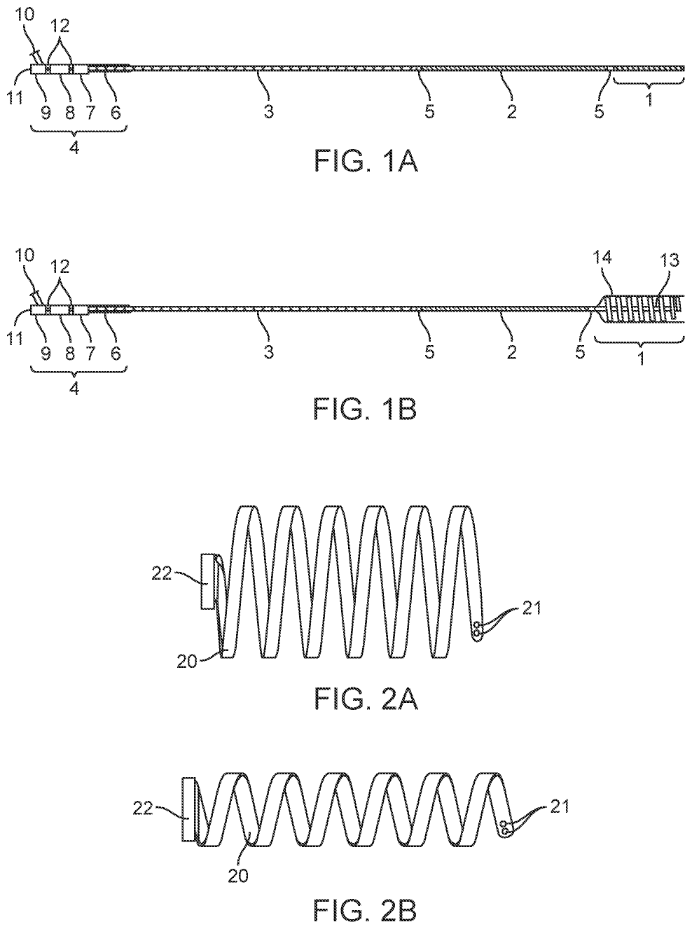

FIG. 1A depicts an aspiration catheter constructed in accordance with the principles of the present invention. The device comprises a distal expandable segment 1, the intermediate segment or intermediate shaft 2, the proximal segment or proximal shaft 3, and the handle 4. The shafts are joined to each other at bonds 5 and to the handle at a strain relief bond 6. The handle comprises a distal handle section 7, a rotating middle handle section 8, and a proximal handle section 9. The distal handle section or the proximal handle section has an aspiration port 10. The proximal end of the device has a lumen 11, which may be configured to receive a guidewire and/or used for aspiration. The middle handle section can rotate relative to the other parts of the handle due to the presence of Swivels 12. FIG. 1A illustrates the distal expandable segment in the collapsed state as it would be introduced into the body and delivered to the target vascular, while FIG. 1B illustrates the distal expandable segment 1 in the expanded state as would be used during aspiration of the clot. FIG. 1B also shows the inner torque member 13 by which the distal expandable segment is expanded and collapsed, and the vacuum resistant membrane 14 which covers the distal expandable segment and connects to the non-expandable sections of the device in order to provide a continuous vacuum path and prevent leakage under vacuum, which could compromise device effectiveness.

The distal expandable segment 1 comprises an expandable and contractible structure which in the contracted state provides a low distal segment profile for superior deliverability, and in the expanded state increases the distal section diameter for improved aspiration. In the preferred example, the distal expandable segment has an outer diameter in the delivery configuration of 2 mm of less, preferably 1.5 mm or less, and most preferably 1 mm or less, and is preferably also less than the outer diameter of the intermediate segment 2 to which it is attached. The distal expandable segment is capable of expanding to a diameter equal to or larger than the clot and/or the vessel occluded by the clot. In the preferred example the scaffold engages the inner wall of the blood vessel to prevent blood leakage past the end of the scaffold when vacuum is applied. The scaffold may be designed to expand such that only the desired portions of the expanded scaffold engage the vessel wall, as desired to balance aspiration performance and risk of vessel trauma. For example only the distal portion of the scaffold may engage the vessel wall, or only the proximal portion, or only a middle section. The scaffold may be intended to be expanded immediately adjacent to the clot or some distance proximal to that. Upon application of vacuum pressure the clot is then drawn into the aspiration lumen of the device.

The distal expandable segment may be configured to expand to a diameter between 2 and 6 mm, more preferably from 3 to 5 mm, and most preferably from 4 to 4.5 mm. Therefore the device of the present invention provides an aspiration lumen in the expandable segment with a cross-sectional area between 1.5× and 10× higher than a conventional aspiration catheter with a fixed diameter aspiration lumen in the 1.4-2.0 mm range. Since the vacuum force applied equals the vacuum pressure times the cross-sectional area, the vacuum force capable of being applied by the device of the present invention is 1.5× to 10× higher than that provided by conventional aspiration catheters, with concurrently superior clot extraction capabilities.

In the example shown in FIGS. 1A and 1B the distal expandable segment 1 is constructed from a single coil structure, the proximal end of which is attached to the catheter intermediate segment 2 and the distal end of which is attached to an inner torque member 13 running through inside of the single coil and attached to the handle 4. Rotation of the inner torque member via the handle causes the coil to be either wrapped tighter thereby decreasing its diameter for delivery (as depicted in FIG. 1A), or to unwrapped to increase its diameter for aspiration (as depicted in FIG. 1B).

The inner torque member 13 may be a solid wire, a tube, or a composite structure such as a polymer shaft with an embedded coil or braid, or a combination thereof. It will typically be as small as possible in order to maximize the area of the aspiration lumen in which it is contained, since a larger inner member occupies more space in the lumen and may negatively affect aspiration efficiency. Solid wires or mandrels of stainless steel, nickel-titanium, or cobalt chrome alloys are most suitable for this application due to having the greatest torque to profile ratio. Ideally such solid members would decrease in diameter towards their distal end in order to minimize impact to system flexibility. However the inner torque member may be tubular and sized to accommodate a guidewire, rather than requiring the guidewire to run adjacent to the inner member and through the vacuum lumen. To minimize wall thickness for flexibility and minimize occlusion of aspiration lumen area while maintaining excellent torque transmission, a tubular inner torque member may be a spiral cut hypotube, with a thin polymer jacket to prevent unspooling when torqued. An inner torque member may comprise more than one of the examples described above, such as a tapered wire in the distal segment which connects to a tubular member more proximally.

FIGS. 2A and 2B show a close-up of a distal expandable segment consisting of a single coil structure, as shown in the expanded state (FIG. 2A) and in a substantially collapsed state (FIG. 2B). The single coil comprises a coil ribbon 20 and may optionally feature distal holes 21 and/or a proximal ring 22 for improving ease of attachment to the inner torque member and catheter intermediate shaft respectively.

The coil structure may be designed in a variety of ways in order to achieve the functional requirements of the device to (i.) deliver to the site of treatment, (ii.) smoothly expand from the collapsed state to the expanded state, (iii.) maintain the lumen shape and resist collapse forces during application of vacuum for aspiration, (iv.) smoothly collapse from the expanded state, and (v.) withdraw the device from the site of treatment.

The coil ribbon 20 may be manufactured from round wire or flat ribbon. Round wire coils would typically be made by wrapping wire around a mandrel and then removing the mandrel, while flat ribbon coils would typically by made by laser cutting a hypotube. Flat ribbon coils may also be wound from flat ribbon wire. Coils may be manufactured from any materials of sufficient strength, flexibility, and biocompatibility for the application. In the exemplary example the coils are made from stainless steel, cobalt chrome, nickel-titanium (NiTi), or titanium alloys. For the same dimensions, stainless steel and cobalt chrome coils provide better torque response than nickel-titanium, but NiTi coils have superior flexibility and are less likely to be damaged during manufacture or use. Coils may also be manufactured from high strength polymers including PEEK, polyimide, and select nylons, polyurethanes, and PETs.

Nickel-titanium (NiTi) alloys in particular are desirable since the super-elastic material is very resistant to kinking and fracture, and also because the NiTi coils and others made from shape memory materials can be heat set into a desired shape. Coils may be heat set into a cone, flared cone, stepped shape, exponential taper and other shapes in order to improve clot engagement and/or coil expansion dynamics. In a preferred example, the distal end of the coil is substantially cylindrical in shape, and the proximal end of distal expandable segment tapers smoothly down to the catheter shafts. The coil may also be heat set to be smallest at the distal end and get progressively larger to the proximal end, or largest at the distal end and get progressively smaller to the proximal end, or even heat set such that in the expanded state it is largest in the middle with smaller ends, or the reverse in which the middle is smallest and the ends are largest. Such heat set geometries play an important roll in coil expansion, and can be used to ensure consistent expansion performance in tortuosity and to prevent twisting of the vacuum resistant membrane covering the coil during expansion. The heat set process can also be used to alter the neutral state of the coil (similar to using a hypotube of a different diameter) and to control spacing between loops of the coil. The coil may also be heat set into an oblong or oval cross-section (when viewed from the end-on) rather than maintaining a circular lumen. This results in a coil of variable profile with a tendency to intermittently lift the vacuum resistant membrane during expansion, reducing potential clinging and twisting of the membrane.

In the exemplary example the coil is constructed from a laser cut hypotube, such that a variety of design attributes come into play. First, the starting tube diameter determines the neutral properties of the coil—larger tubes result in a coil with more strength and uniformity in the expanded state but may be more difficult to collapse to a low profile. The tube and therefore coil ribbon wall thickness also significantly impacts the strength, flexibility, collapsibility, and radiopacity of the coil. Tubes suitable for this application are typically in the range of 1.0 to 3.5 mm outer diameter, with a wall thickness of 0.0015″-0.004″. Thicker tubes up to 0.008″ wall thickness may also be suitable, especially if significant material may be removed during processing such as electropolishing. Depending on the designed geometry, laser angle, and electropolishing process (if any), the coil cross-section geometry can be circular, square, rectangular, trapezoidal, etc.

The length of the coil structure and hence of the distal expandable segment may be as desired. In the exemplary example the length may be as short as 1 mm or as long as 150 mm. Shorter elements require fewer rotations to open yet still provide the full increased tip vacuum force of the present invention, while longer distal expandable segments create a larger chamber to take in and hold larger/longer and more fibrous clots which need to be pulled out intact. The length of the distal expandable segment may also impact deliverability.

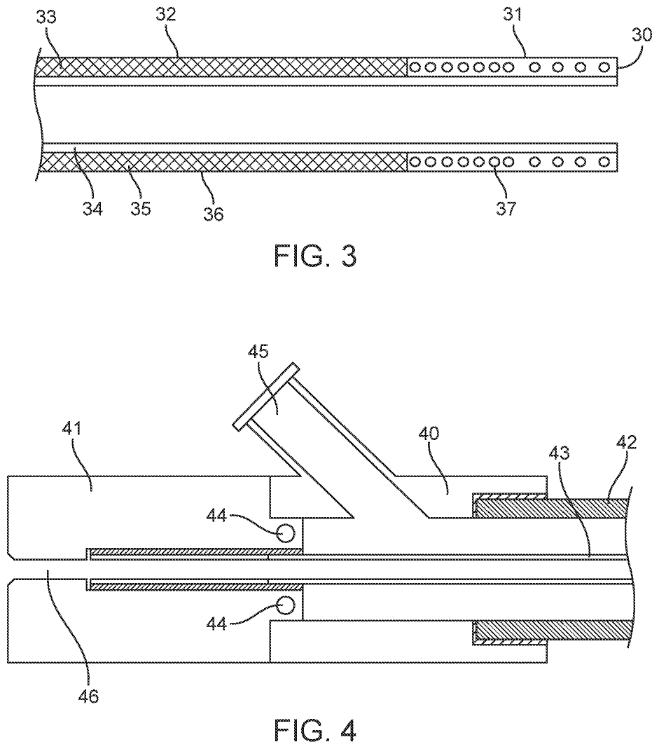

FIG. 3 shows a close up of one example of the catheter outer shaft, comprising an elongated tubular body with a distal end 30, an intermediate outer shaft 31, a proximal outer shaft 32, and a proximal end 33. The purpose of the catheter shaft is to allow the distal expandable segment to be advanced to and withdrawn from the target area, to permit torque to be transmitted from the handle mechanism to the distal expandable segment so it can be expanded and collapsed, and to provide a fluid-impermeable lumen through which vacuum pressure can be applied to the distal end of the device. In the exemplary example, the catheter comprises two segments with distinct properties, the intermediate shaft (segment) and the proximal shaft (segment), although variants with more than two shaft sections are envisioned and may be superior for some applications.

In general, the proximal outer shaft 32 of the device runs from the user-operated handle on the proximal end of the device (and outside the patient's body) through the femoral artery access point, up the aorta, and into the base of the carotid or vertebral arteries. The proximal outer shaft will be firmer than the intermediate segment and optimized for torque and/or linear force transmission. The intermediate segment of the device will be optimized for flexibility such that the distal segment and the intermediate segment can be tracked through the tortuous intracranial neurovascular anatomy to the site of the clot. The intermediate segment must retain sufficient torque and/or linear force transmission capability to allow the distal expandable segment to be expanded and collapsed.

A variety of metal and polymer technology well known within the industry may be used to manufacture the catheter shafts. In the exemplary example proximal outer shaft 32 comprises lubricious polymer inner liner 34, a metallic or polymeric braid 35 in the core, and a firmer polymer outer jacket 36. The inner liner is typically made from PTFE, FEP, HDPE, or another lubricious polymer to allow the underlying inner member or guidewire to rotate smoothly, the braid is made from stainless steel or nickel-titanium alloy to provide strength, kink resistance, and efficient torque transmission, and the outer jacket is made from Polyether block amide (Pebax®), nylon, polyether ether ketone (PEEK), or polyamide. In the exemplary example the intermediate outer shaft will be of similar construction to the proximal outer shaft, except the core layer will contain an embedded support coil 37 rather than a braid in order to maximize the flexibility of this portion of the shaft while maintaining lumen integrity and prevent kinking around tight corners. The outer jacket of the intermediate outer shaft will also be manufactured from softer and more flexible materials like low durometer (25 D-55 D) Pebax or similar. The embedded support coil may be a spring guide in which the adjacent loops of the coil are in direct contact with each other in order to provide maximum axial stiffness, shaft pushability, collapse resistance, and radiopacity.

For single coil and some other device designs, it may be advantageous in an example to use a multilumen shaft design in the intermediate and/or proximal segments where the largest lumen is used for aspiration, and the smaller lumen(s) used for guidewire passage, contrast injection, or to sequester the inner torque member. This provides a continuous and unobstructed aspiration lumen which may aspirate clot more effectively than a lumen partially occluded by one or more objects inside of it.

Any elongated tubular member can be shaped into an accordion or convoluted form to increase flexibility. The accordion or convoluted form can also reduce surface contact to minimize surface friction between different moving components within the system or between the elongated tubular member and the wall of the blood vessel.

FIG. 4 shows a close up of one example of the device handle comprising a fixed handle body 40 and rotating handle knob 41. The catheter shaft proximal segment 42 attaches to the fixed handle body, while the inner torque member 43 attaches to the rotating handle knob. Smooth rotation of the handle knob is facilitated by ball bearings 44. The handle body contains an aspiration port 45, while the entire assembly preferably has an inner lumen 46 for guidewire passage.

The handle mechanism connects to both the inner and the outer members of the proximal shaft and allows the physician to rotate one with respect to the other, thereby transmitting torque to the intermediate segment and expandable distal segment. In the exemplary example, the outer member is fixed and only the inner member rotates such that the outer member is stationary versus the vessel wall for minimal vessel trauma, although the reverse is envisioned, as is a variant in which both shafts are rotated simultaneously.

The handle may be designed for manual operation, with the inner and outer members connecting to different elements of the handle with a swivel between them to maintain integrity and alignment. The handle may contain a gearbox mechanism to reduce the number of turns needed by the physician to expand the expandable distal segment. The handle may also incorporate a motor which eliminates the need for manual manipulation. In some design examples, the proximal ends or toward the proximal ends of the elongated tubular member(s) and/or torque elements may terminate in simple proximal hubs, allowing the physician more freedom of operation. Such hubs may incorporate side-arms for aspiration, luer locks to keep all parts in position during device advance and/or withdrawal, and/or Tuohy-type hemostatic valves to anchor guidewires or microcatheters and to minimize blood loss during the procedure.

In another example, the handle is designed such that the inner torque element attached to the distal end of the coil is held fixed and the outer shaft is torqued to rotate the proximal end of the coil, thereby tending to unwrap and expand the coil from a substantially proximal to distal direction. This design may provide superior expansion performance in tortuous anatomy.

In another example, the handle also causes the inner torque element attached to the distal end of the coil to move distally and proximally instead of or in addition to rotating the coil to cause it to collapse or expand. Distal movement of the inner member causes the coil to lengthen and collapse in profile, while proximal movement of the inner member causes the coil to shorten and expand in profile. This approach may provide superior expansion performance in tortuous anatomy and allow for an overall lower profile of the fully collapsed device.

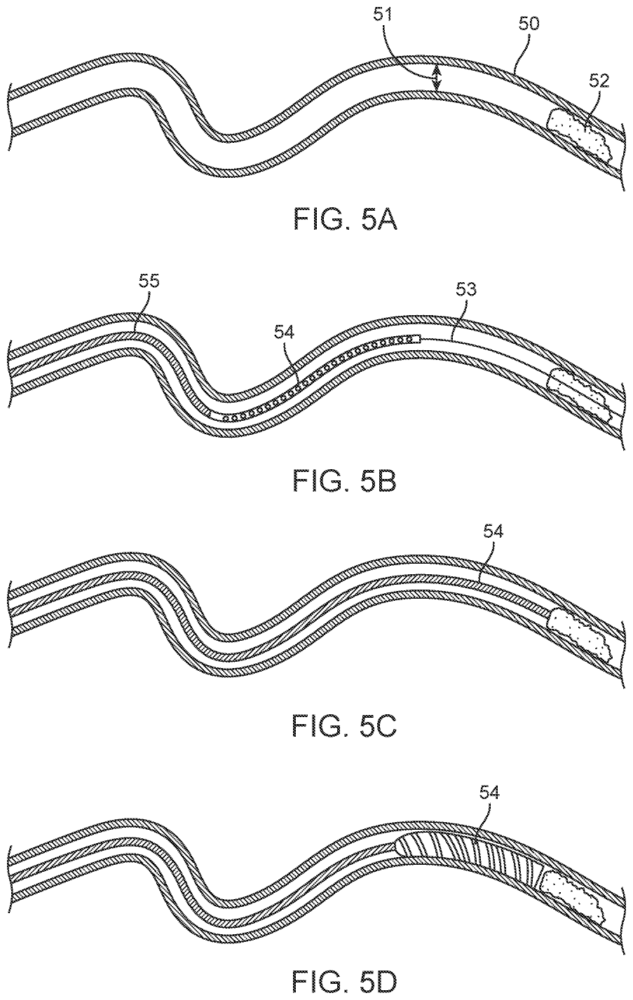

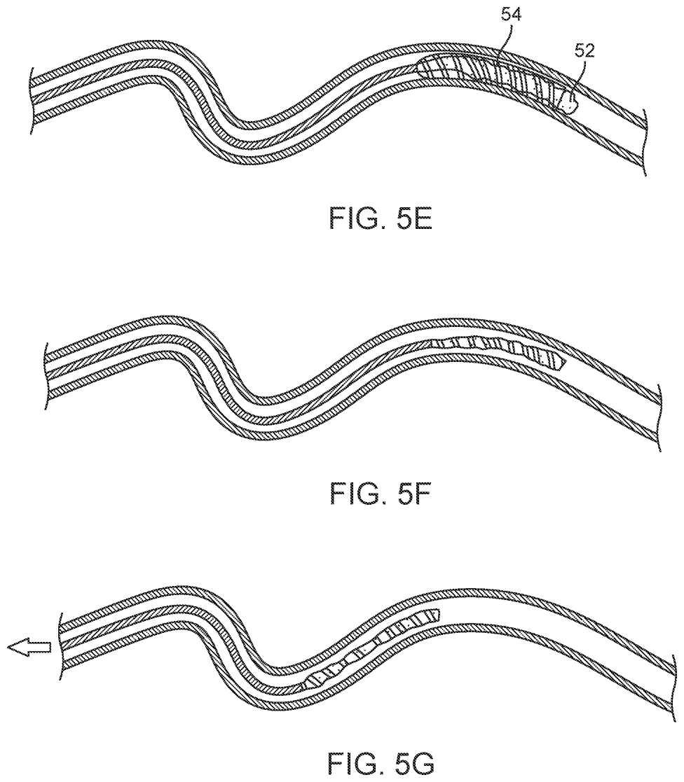

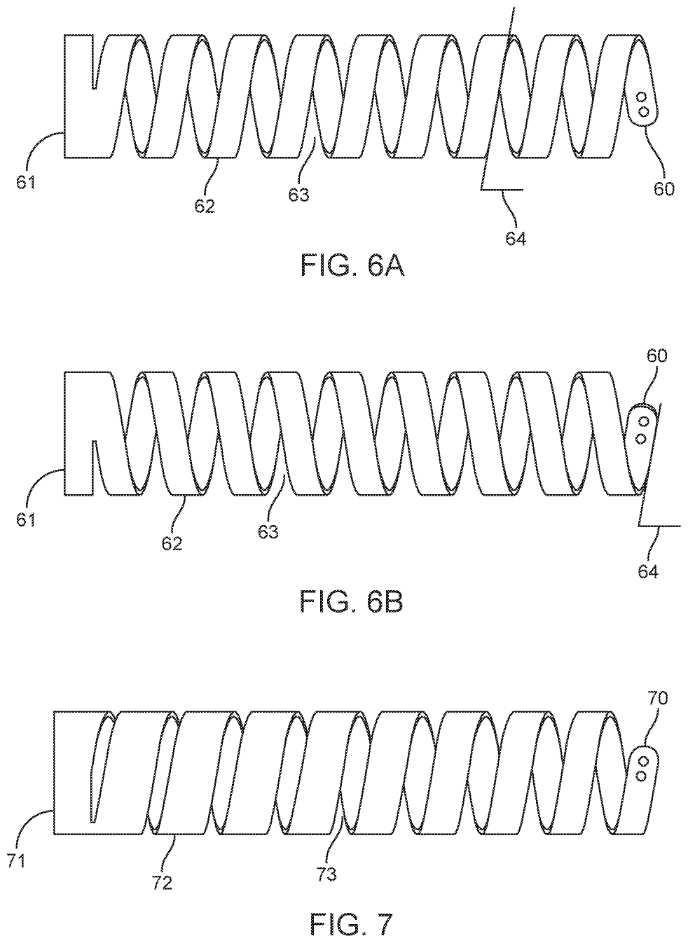

FIGS. 5A-5G depict an example of the device of the present invention in use.

FIG. 5A shows the anatomy as the patient presents to the physician, consisting of a blood vessel 50 with an inner lumen 51 which is occluded by a clot 52.

FIG. 5B shows the next steps in the procedure, in which a guidewire 53 has been advanced through the blood vessel inner lumen in through the clot, thereby providing a rail upon which the device of the present invention can be advanced. In the figure, the device including the distal expanding segment 54 in its collapsed state and the intermediate segment 55 have been threaded over the guidewire and advanced into the vascular anatomy.

FIG. 5C shows the next steps in the procedure, in which the device has been advanced until the collapsed distal expanding segment 54 is adjacent to the clot, and the guidewire has been withdrawn.

FIG. 5D shows the distal expanding segment 54 after expansion and prior to application of vacuum pressure to aspiration the clot.

FIG. 5E shows the distal expanding segment 54 after aspiration, wherein the clot 52 has been pulled within the distal expanding segment. Ideally the clot would be broken up during the aspiration process and fully removed from the body, but aged and/or fibrous clots may be exceptionally cohesive and may need to be physically pulled from the anatomy by the device, as shown.

FIG. 5F shows the distal expanding segment 54 after it has been recollapsed to trap any clot which was not fully aspirated through the device.

FIG. 5G shows the device as it is being withdrawn from the anatomy with any remaining clot trapped within it.

### Coil Variants

There are numerous aspects of the coil design which can be used to optimize its performance in particular anatomies and/or in conjunction with other parts of the system such as the inner torque member and the distal sleeve. In particular the performance of the coil will depend on the direction of coil wind, ribbon width, pitch angle, gap between ribbon loops, and number of ribbons in the wind. These design attributes may be constant along the length of the coil, or vary to provide improved collapsed or expanded properties.

FIGS. 6A and 6B show an example of a standard coil with a distal end 60, a proximal end 61, ribbon 62, ribbon gap 63, and pitch angle 64. In this example the ribbon width, pitch angle, and ribbon gap distance are constant through the length of the coil such that in absence of other factors the coil will tend to expand simultaneously and uniformly along its length. Ribbon width will typically range between 0.008″ and 0.065″. Wider ribbons result in stronger coils which resist collapse from vacuum pressure better, but they are stiffer and less deliverable than narrower ribbons. The thickness of the coil ribbon also impacts these properties. In one example the ribbon width is about 0.030″ and thickness is about 0.002″, resulting in a 15:1 ratio of width to thickness.

The coil structure helix will typically have a pitch angle 64 in a range from 50° to 85° from the longitudinal axis. Higher pitch angles result in more loops per linear length and generally less gap when the coil is expanded but require more rotations to open. The pitch angle can be determined at laser cutting, or, for NiTi coils, at heat set. In one variant of the design, the distal loops of the coil are heat set into a 90° angle such that they provide an aspiration lumen mouth that is perpendicular to the vessel axis. Such loops can be stacked for greater radial strength and may or may not overlap when in the collapsed state. The coil loops can be cut or heat set into a reverse angle in parts or all of the coil, such that the contact between the coil and the sleeve varies as the coil opens.

In the fully collapsed state there is typically little or no ribbon gap 63 between ribbon loops. Depending on ribbon width, expansion diameter, and length change allowed, the gap between the ribbon loops in the expanded state may be less than the ribbon width or up to several times greater than the ribbon width. Tighter gaps in the expanded state typically correspond to designs which allow the expandable element to shorten during expansion. Gaps between ribbon loops in the collapsed state can also increase flexibility for improved device deliverability, and/or be used to influence expansion, particular with respect to promoting distal sleeve stretching or unfolding when around a bend in vascular tortuosity.

FIG. 6A shows the ribbon with a counter-clockwise wind as viewed from proximal to distal, such that the inner torque member would be rotated counter-clockwise to collapse the coil and clockwise to expand it. FIG. 6B shows the ribbon with a clockwise wind as viewed from proximal to distal, such that the inner torque member would be rotated clockwise to collapse the coil and counter-clockwise to expand it. The direction of rotation matters primarily from an intuitiveness and ergonomic aspect for manually-operated handles. For an physician operating the handle with his right hand, it is most intuitive to rotate the handle knob clockwise to expand the distal expandable segment, such that a counter-clockwise ribbon would be used if there is a direct connection from the handle knob to the inner torque member. If the handle contains gearing which causes the inner torque member to rotate in a direction opposite to the direction in which the handle knob is turned, then a clockwise ribbon wind would be used to maintain the clockwise knob rotation for device expansion.

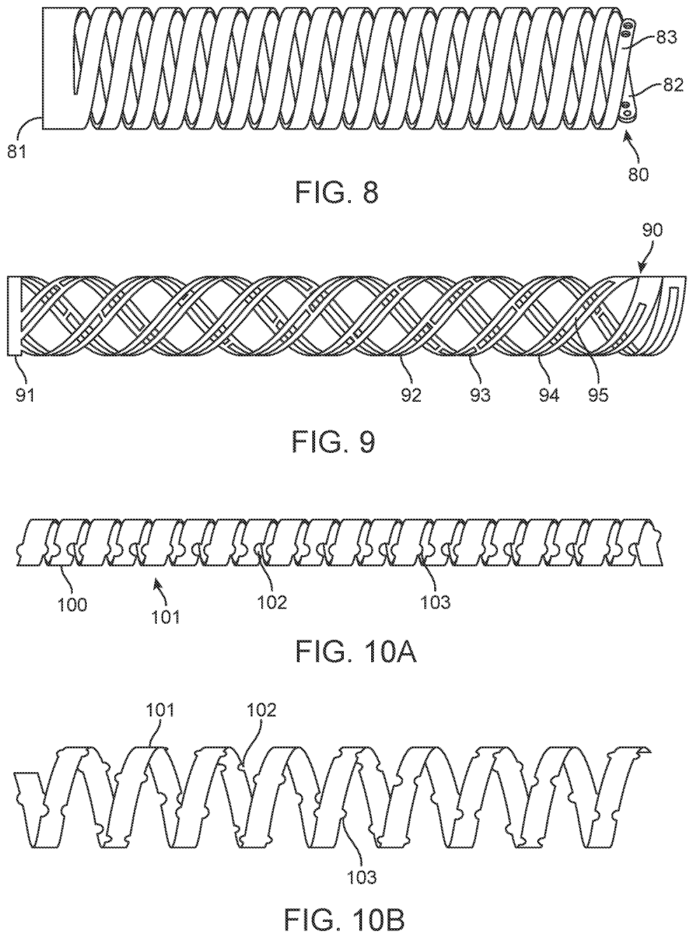

FIG. 7 shows an example of a coil with variable ribbon width featuring a distal end 70, a proximal end 71, ribbon 72, and ribbon gap 73. In the example illustrated, the ribbon width decreases from 0.040″ at the proximal end (20:1 width to thickness ratio on a 0.002″ tube) to 0.020″ at the distal end (10:1 ratio). Because the pitch angle of the ribbon width is constant, the ribbon gap increases from proximal to distal as the ribbon width narrows. Alternatively, the increase in width can progress in the opposite direction from proximal to distal end. Variance in coil ribbon width may be linear with length or non-linear, such that the increase or decrease in width occurs more or less rapidly down the length. Such variances in ribbon width can significantly impact coil flexibility and expansion, particularly in combination with the coil pitch angle and any taper to the heat set coil. Used separately or together, these features may promote coil opening and/or reclosing from distal to proximal, proximal to distal, or evenly, and be used to balance the impact of the presence of the distal sleeve on coil expansion. In example shown with the distal end of the coil having a narrower ribbon than the proximal end, the device will be slightly more trackable through the anatomy due the progressively increasing flexibility towards the distal end, and it will also seek to expand first at the distal end and propagate proximally. Depending on other factors, the final expanded coil will tend to have a slightly tapered shape in the expanded state, being larger at the distal end and smaller at the proximal end.

FIG. 8 shows an example of a double helix coil with a distal end 80, a proximal end 81, a first ribbon 82, and a second ribbon 83. Whereas a coil with a conventional spring construction consists of a single ribbon spiraling helically around the center axis, this Figure illustrates a double helix construction (i.e. like DNA) with two the parallel ribbons 82 and 83 spiraling about the center axis. Other examples have three or more helixes. In general more helixes provide greater coverage such that pitch helix angle (versus the axis) is decreased, resulting in fewer winds necessary for a given length and fewer rotations at the distal end needed to expand/collapse the coil, at the cost of potentially decreased flexibility.

FIG. 9 shows an example of a triple helix coil with a distal end 90, a proximal end 91, a first ribbon 92, a second ribbon 93, and a third ribbon 94 in which all ribbons feature slots 95 cut into the core of the ribbon, thereby creating a coiled ladder structure. Adding slots to the ribbon of any coil may provide varying contact area against the distal sleeve for improved expansion. A ladder structure to the ribbon would also allow for a coil with wider ribbons which would remain flexible in the crimped state but would be more resistant to axial elongation and rotational distortion, potentially enabling designs with shallower pitch angles and more spirals to reduce the number of rotations needed to expand the coil.

FIGS. 10A and 10B shows an example of a collapsed coil 100 and expanded coil 101 featuring laser cut notches 102 and bumps 103. The edges of the coil ribbon may be laser cut into contours such as waves, bumps, notches, or other geometric features. As the coil expands and the loops rotate past each other, these features provide varying contact area against the distal sleeve to reduce propensity of the sleeve to twist and to enhance expansion uniformity.

In another example, the coil is a radially expandable separator scaffold extending distally from the distal end of the catheter body and includes helically arranged cutting elements which define a central clot-receiving passage. The separator scaffold may feature a smooth ribbon profile, or contoured edges of the type shown in FIGS. 10A and 10B. The separator scaffold may be radially expanded in the blood vessel and rotated and advanced to resect clot. The aspiration lumen of the catheter body and the central clot-receiving passage of the radially expandable separator scaffold are arranged coaxially so that clot resected by rotating the separator scaffold may be aspirated into the aspiration lumen of the catheter body by applying a vacuum to a proximal end of the aspiration lumen. The separator scaffold may also be used to press clot against the vessel wall and/or squeeze it within the coils as may be desirable to disrupt the clot prior to or during aspiration.

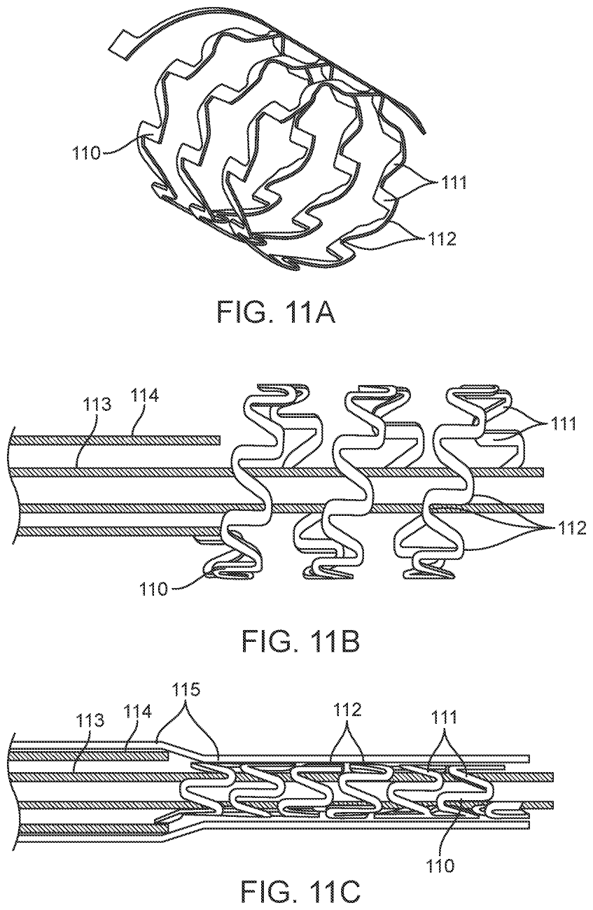

FIG. 11A shows an example of a coil with a sinusoidal ring ribbon 110 comprising struts 111 joined by crowns 112. FIG. 11A shows an oblique view of the stand alone coil. FIG. 11B shows a side cross section of the distal end of the device in the expanded configuration showing the sinusoidal ring ribbon 110 along with the inner torque member 113 and intermediate outer segment 114. FIG. 11C shows a side cross section of the distal end of the device in the collapsed configuration including the presence of a constraining sleeve 115. (In FIGS. 11B and 11C the vacuum resistant membrane which would normally cover the coil and distal end of the intermediate outer shaft has been omitted for clarity).

The main advantages of this example is that in addition to conventional winding/unwinding to expand/collapse the coil, the sinusoidal ring of the present example can itself can expand in length, thereby assisting in expansion of the structure. The effectively wider width of the ribbon of the sinusoidal coil may also provide benefits with regards to supporting the distal sleeve during vacuum application.

In one example the sinusoidal ring ribbon 110 is made from nickel-titanium or other shape memory material cut into a sinusoidal pattern and heat set with the sinusoids open and the coil ribbon in the expanded position, such that the sinusoids are pressed into a closed position when the device is compressed into the collapsed state. The coil is then sheathed, capped, or otherwise captured in the constrained state. After delivery to the site of treatment, the sheath or cap is removed allowing the sinusoids to open to increase the diameter of the expandable segment, after which the coil can then be torqued normally to provide additional diameter control. In another example, the sinusoidal ring coil is made from a polymer which seeks to expand when exposed to moisture and/or heat. Such materials typically take a few minutes to fully expand, such that no constraint method is needed other than through torque control at the ends of the coil. The device of this example is advanced to the site of treatment, then the coil is torqued to expand it to contact the vessel, and then as the material further warms and hydrates it will seek to expand further, improving the seal against the vessel to prevent blood leakage during aspiration. After aspiration, the sinusoidal ribbon coils are fully or partially collapsed by applying a torque to them as has been described previously with non-sinusoidal coil designs.

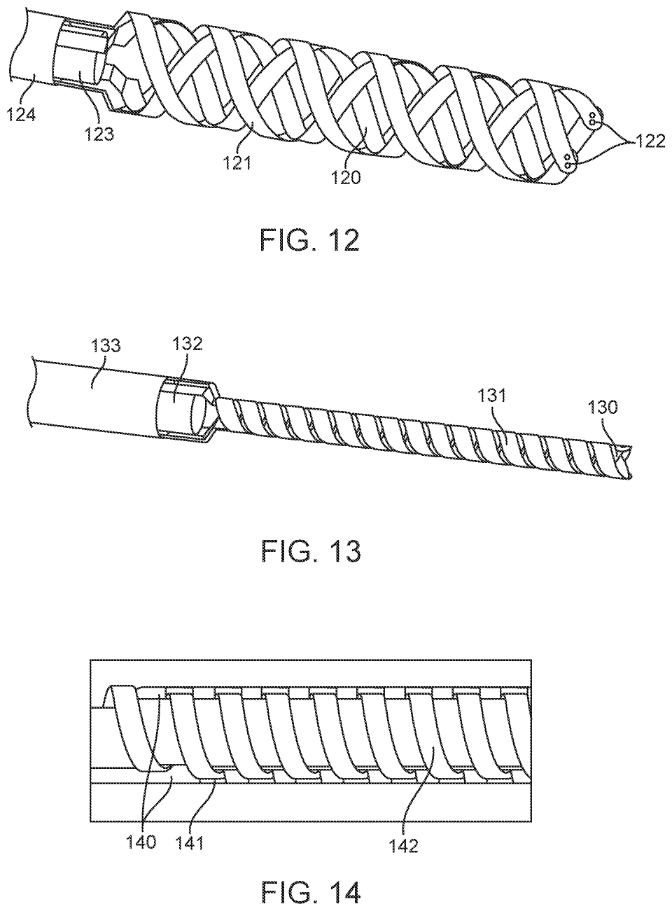

FIGS. 12 and 13 illustrate a preferred example of the present invention, in which the distal part of the inner torque member has been replaced with a second inner coil 120, 130 wound in direction opposite to that of the outer coil 121, 131. FIG. 12 shows the dual coil system in the expanded state, and FIG. 13 shows the dual coil system in the collapsed state. The two coils are joined at their distal ends 122 and hence act in unison. The two coils in the dual coil design may be attached to each other using a variety of techniques including welding, crimping, wrapping/tying with a strap or wire, rivets, or with a tab-and-slot interface. In this configuration, one coil torques against the other (usually the inner against the outer) causing both coils to open. In the neutral state the outer coil is of larger diameter than the inner coil and an optimal clearance between the two is maintained to achieve smooth rotation and the desired diameter changes.

The remainder of the catheter is substantially the same as previously described, except that the inner torque member 123, 133 terminates at approximately the end of the intermediate segment where it is then bonded to the proximal end of the inner coil. The inner torque member (of both the intermediate and proximal shafts) will typically be as large as possible in order to maximize the vacuum lumen area which lies within, and where any guidewires or supplementary devices will be tracked. Size of the proximal and intermediate inner member will be limited by the inside diameter of the outer member 124, 134 and the clearance needed between the two to allow smooth rotation and expansion and collapse of the distal expandable segment.

While the single coil example has the advantage of simplicity of manufacture, potentially lowest profile, and increased distal robustness in the collapsed state which may aid in delivery (especially if the torque element is tubular and sized to accommodate a guidewire which the device may be tracked along), the torque element takes up space in the aspiration lumen which reduces the effective tip surface are and vacuum force that can be applied. Depending on the stiffness of the inner torque element, the single coil example may also be less deliverable. In comparison, the main advantages of the dual coil example are greatest flexibility in the distal expandable segment due to the absence of any solid wire or tubular element therein, and maximum tip area in the expanded state.

The coils in the dual coil system are preferably made from NiTi due to its superior robustness, and also because NiTi is heat treatable which provides an easy-to-manufacture means of obtaining tapered coils. Tapered coils may be of benefit in achieving ideal spacing between the inner and outer coils and ensuring smooth expansion/contraction of the distal segment. In the exemplary example of the dual coil design, both the inner and outer coils are heat set to impart a tapered or conical shape, with the distal end of the coils being larger in diameter than the proximal end by about a 1.5:1 ratio. Typically, the outer coil and inner coil of such a dual coil design are heat set into different tapers intended to control spacing and friction between the two during expansion.

The coils in a two coil system may differ with respect to coil ribbon thickness, ribbon width, pitch angle, ribbon gap, etc., and either or both coils may utilize any of the other features and variants previously described, such as variable ribbon widths, multiple helixes, edge contours, sinusoidal rings.

FIG. 13 also illustrates a major advantage of the design of the present invention, which is that when the distal expandable segment is in its collapsed and constrained state it may be of significantly smaller profile than the intermediate segment with the fixed diameter aspiration lumen, thereby allowing for improved deliverability with less vessel trauma.

FIG. 14 shows another example of the present invention in which tubular attachments 140 with slots 141 are employed to maintain a constant spacing of the coil ribbon 142 through diametric changes. The slots have widths sufficient to fit the width of the coil ribbon. The slots are positioned 180 degrees apart in each tube and designed to have the coil spiral enter and exit freely in a radial direction. When fully closed, the coil tightly wraps to the tube diameter. When fully open, the coil spiral diameter increases yet spacing between loops is maintained by the tube attachment. Alternately a single slotted tube can be added to further control loop spacing of the expanded coil.

In alternate example of the coil design of the present invention, the distal end of the coil is attached to a wire, tube, other member located outside of the coil and the proximal end of the coil is attached to the catheter shaft. The outer member runs the length of the device such that torque applied to the proximal end of the outer member is transmitted to the distal end of the coil, thereby causing it to rotate to expand or collapse. If the outer member is tubular, it can serve as a secondary lumen for contrast injection, guidewire passage, or other purposes.

In an alternate example of the coil design of the present invention, a wire, tube, other member is located outside of the coil, with the distal end of this member attached to the distal end of the coil and the proximal end of this member is attached to the distal end of the intermediate segment. The proximal end of the coil is then attached to a rotating tubular torque element inside the intermediate segment outer member, such that the coil is rotated from its proximal end while the distal end is held fixed. This arrangement promotes coil expansion in the tight tortuosity, and furthermore the wire, tube, or other member running outside the coil provides an anchor for the vacuum resistant membrane. If the design features a tubular member running outside the coil, the tubular member can extend to the proximal end of the device and serve as a secondary lumen for contrast injection, guidewire passage, or other purposes.

In another example of the single coil design of the present invention, the device shafts comprise 3 elongated tubular members running the length of the device. The innermost elongated tubular member attaches to the distal end of the coil, the outermost elongated tubular member attaches to the proximal end of the coil, and the elongated tubular member between the other two tubular members attaches to the single coil somewhere in the middle of the coil. This additional shaft and attachment point allows the distal and proximal sections of the coil to be expanded and collapsed separately, to provide for variable expansion diameters best suited for vessel and clot properties, and/or to assist with distal sleeve expansion without twisting. In an alternate example utilizing the same shaft setup, the distal and proximal sections of the coil have opposite winds, such that the coil can be entirely expanded and collapsed by rotating the middle member attached to the center of the coil while the innermost and outermost elongated tubular members attached to the distal and proximal ends of the coil respectively are held fixed.

### Example 2: Distal Expandable Segment Comprising a Self-Expanding Scaffold

In a preferred example of the present invention, the distal expandable segment comprises a self-expanding scaffold. In one variant of the design, the self-expanding scaffold is in the neutral state when full expanded and is elastically compressed into the collapsed state and then constrained, and re-opens to the expanded state upon removal of the constraint. In another variant of the design, the scaffold naturally remains in the collapsed state without a constraint and only expands upon application of external stimuli such as heat, moisture, electricity, etc.

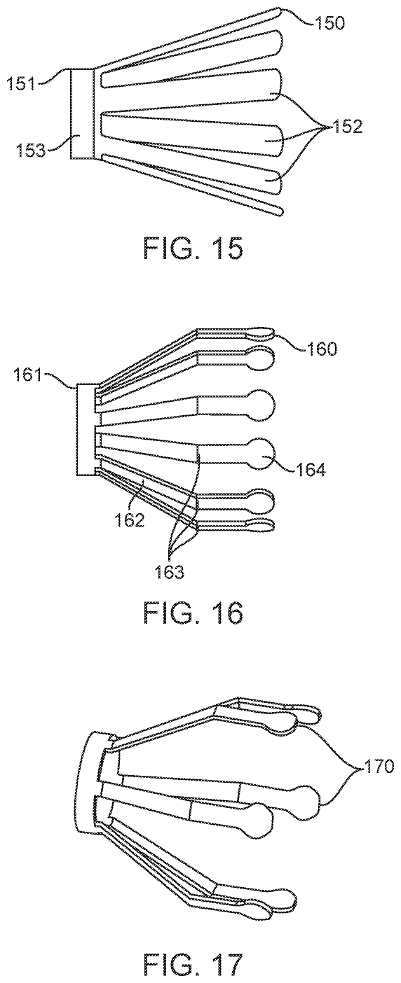

FIG. 15 shows an example of a self-expanding scaffold with a distal end 150 and a proximal end 151, comprising a plurality of struts 152 radiating in a distal direction from a common circular base 153. The base 153 is attached to the elongated tubular body of the catheter shaft. The scaffold is substantially conical in profile with a proximally-oriented apex providing a taper to smoothly channel clot into the smaller lumen of the intermediate segment.

The scaffold may contain between 3 and 20 of the linear struts 152, more preferably between 5 and 12 struts, and most preferably between 6 and 8 struts. The widths of the struts may be the same for all struts in the scaffold, or vary between struts or within struts as designed to affect the profile properties of the scaffold. In one version of this example, the width of the struts can be designed to encircle the circumference of the tube. For example, for a scaffold cut from a tube with an outer diameter of 1.8 mm, thereby having an outer perimeter of 5.65 mm, the scaffold may have 6 struts each of 0.94 mm width. In another version of this example, the struts can have a width less than the maximum allowed by the tube's circumference in order to allow the struts in the self-expanding scaffold to collapse to a crimped configuration smaller than the diameter of the tube from which the scaffold is cut. In a preferred example of the present invention in which the self-expanding scaffold comprises linear struts or struts, the targeted crimped profile is 1 mm in diameter. In a self-expanding scaffold with six linear struts of equivalent width, the width of each strut would be approximately 0.5 mm.

The self expanding scaffold may be of a length from 1 to 10 mm, more preferably from 1 to 5 mm, and most preferably from 2 to 3 mm. Shorter length scaffolds are more trackable through tortuous vessels, while longer length scaffolds will have a lower angle of opening and will funnel clot easier into the aspiration lumen.

The self-expanding scaffold is manufactured such that it will expand to a diameter equal to or larger than the vessel diameter it is intended to treat. The scaffold may be configured to expand to a diameter between 2 and 6 mm, more preferably from 3 to 5 mm, and most preferably from 4 to 4.5 mm. In one preferred example, the expandable scaffold expands to a diameter larger than the adjacent non-expandable segment of the delivery system ranging from 1.1 times to 3 times the non-expandable segment, and more preferably expands from 1.2 times to 2 times the diameter of non-expandable segment. Therefore the device of the present invention provides an aspiration lumen in the expandable segment with a cross-sectional area between 1.5× and 10× higher than a conventional aspiration catheter with a fixed diameter aspiration lumen in the 1.4-2.0 mm range. Since the vacuum force applied equals the vacuum pressure times the cross-sectional area, the vacuum force applied by the device of the present invention is 1.5× to 10× higher than that provided by conventional aspiration catheters, with concurrently superior clot extraction capabilities.

In another example, the self-expanding scaffold is contoured for maximum performance in the desired anatomy. The self-expanding scaffold may be conical, hemispherical, or substantially cylindrical in shape, or may be a combination of the described shafts. Furthermore, the distal edge of the self-expanding scaffold may be further contoured with a flare to increase expansion diameter and aid vessel sealing, or with a taper to minimize vessel trauma during advance or withdrawal of the device.

FIG. 16 shows an example of a self-expanding scaffold with a distal end 160 and a proximal end 161, and in which the struts 162 have bends 163 allowing the expanded scaffold to better conform to the vessel in the expanded state for superior vacuum sealing. The struts also have rounded tips 164 in order to minimize vessel trauma and/or provide a larger surface area for membrane attachment. The radii of curvature of the rounded tips may be one half the width of the strut such that the strut terminates in a semi-circle, or the radii of curvature may be larger such that the struts terminate in oversize rounded tips. In another example, the self-expanding scaffold comprises struts having ovalized ends. In a preferred example, the struts terminate in oversize rounded tips of diameter approximately 1.5 times to twice the width of the strut.

FIG. 17 shows a variant of the above example in which the rounded tips of the struts can have flats 170 on the leading edge to further reduce vessel trauma and/or better distribute loads against the vacuum-resistant membrane. An alternative example the flats are on one or both sides of the tips in order to allow for tighter crimping. In a preferred example, the flat edge length is approximately ¼ to ¾ the diameter of the rounded tip.

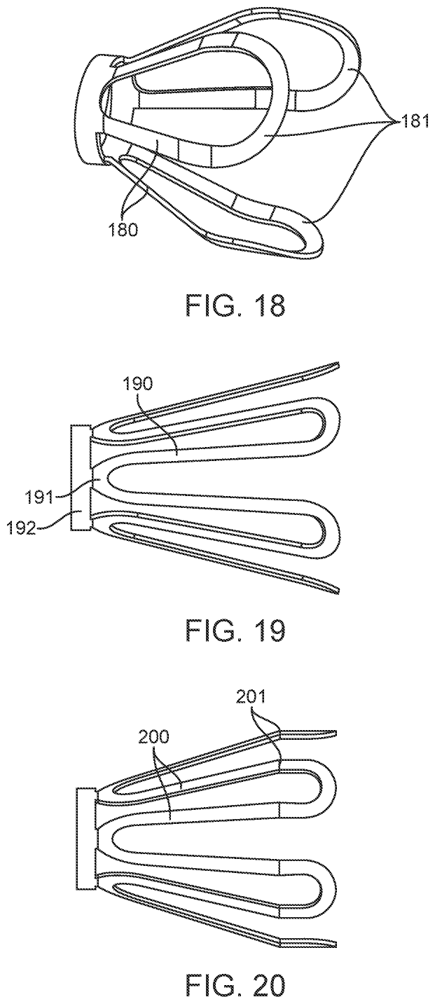

FIG. 18 shows an example of a self-expanding scaffold comprising a plurality of struts 180 and in which two or more struts are connected by arcs 181 to an adjacent strut thereby forming loops. For example, a self-expanding scaffold containing 12 struts could be formed into 6 independent loops, or four wings of 3 connected struts each, two wings of 6 connected struts each, etc. The connected arc angle can be a tangential half circle equivalent to 180 degrees such that the struts remain parallel to the axis. Alternatively, the connecting arc of the loop is designed with an arc angle different than 180 degrees, such that the linear struts used to form each loop are no longer parallel to one another or the axis. A smaller arc would draw the tips of the linear struts together such that the scaffold can crimp to a lower profile at the distal end, or a larger arc (shown) would provide for a larger starting distal profile and possibly improved expansion and clot engagement. The width of each loop and total number of loops in the self-expanding scaffold system can be used to determine a final crimped profile when pressed to contact (in the absence of strut/loop overlap). For example, a self-expanding scaffold with six equal loops of 0.6 mm outer loop width would allow for a 1.2 mm crimped profile to be attained.

FIG. 19 shows a variant of the above example in which the proximal ends of the struts 190 are also connected by arcs 191, thereby forming a sinusoidal ring or serpentine structure. In this example the curved ends, or “crowns”, of the sinusoidal ring structure are connected directly to the base 192. The sinusoidal ring structure may contain from 3 and 12 crowns, more preferably from 4 and 8 crowns, and most preferably from 4 to 6 crowns. The width of the struts in the sinusoidal ring may be between 0.005″ and 0.020″, more preferably 0.006″ and 0.014″, and most preferably 0.008″ and 0.012″. As such the ratio of ring strut width to linear strut width (when the latter are present) may vary from approximately 0.5:1 to 2:1. A flat strut may added to each crown apex of the sinusoidal ring feature in order to convert bending stress into compressive stress to enhance fracture resistance of the sinusoidal ring. In another example, the crowns at the distal end of the scaffold have a larger radius of curvature than the crowns at the proximal end of the scaffold such that the struts of expanded scaffold more gradually taper to the shaft.

FIG. 20 shows a variant of the above example in which the struts 200 have bends 201 near the crown tips allowing the expanded scaffold to better conform to the vessel in the expanded state for superior vacuum sealing.

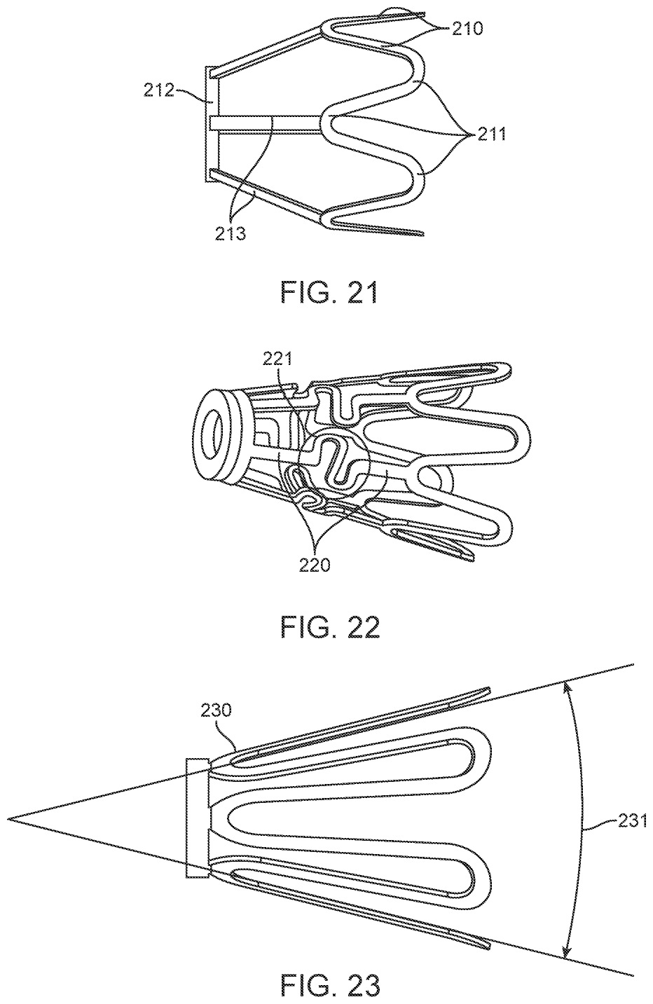

FIG. 21 shows an example of a self-expanding scaffold comprising a plurality of struts 210 connected by arcs 211 at both ends to form a sinusoidal ring structure, and in which the proximal end of the ring is attached to the scaffold base 212 by linear strut links 213. This design allows the sinusoidal ring to more evenly share compression load between the distal and proximal crowns thereby increasing the expansion force and resistance to vacuum collapse. Furthermore the sinusoidal ring aids in maintaining a circular entrance to the aspiration lumen.

The sinusoidal ring axial length may be from 30% to 60% of the total scaffold length, more preferably from 40% to 50% of the total scaffold length. For example, if the total length of a self-expanding scaffold is 5 mm, the sinusoidal ring may be 2 mm and the linear struts connecting it to the elongated tubular body may be 3 mm.

In the preferred example, each proximal-facing crown tip in the sinusoidal ring scaffold is anchored by a linear strut link to prevent unanchored crown tips from interfering with sheath advancement or from potentially inducing vessel trauma during device pullback in the expanded state. In another example, the sinusoidal ring scaffold has more crowns than there are linear struts, allowing for greater scaffold flexibility for device delivery in the patient. In an alternate example the links connect to the middle of the struts in the sinusoidal ring or to the distal end crowns rather than to the proximal crowns.

In another example, the links are not coaxial with the centerline of the elongated tubular body and wrap in a spiral configuration to improve system flexibility or evenness of expansion in tortuous anatomy. For instance, the base of the link can be aligned to one crown of the sinusoidal ring with the ring attachment at the adjacent ring crown. Alternatively, the wrapping angle is increased by further offsetting the link attachment to the next adjacent ring crown. In another example, one or more of the links attaching the sinusoidal ring to the scaffold base are split through the axial length producing a sinusoidal ring having multiple crown members. This configuration reduces rigidity of the self-expanding scaffold to aid vessel conformability during track and expansion.

In an alternate example of the present invention, the scaffold may be composed from more than one sinusoidal ring attached to each other and/or the catheter shafts directly and/or with straight, curves, or articulated links. In a parallel design well suited for ease of manufacture, a tube is cut with alternating slots in order to create a structure of conjoined serpentine rings in the expanded state, in a pattern well known to those in the industry.

FIG. 22 shows a variant of the above example in which the links 220 include a ‘U’, “M”, ‘Z’ or ‘S’ or similar geometry 221 in order to increase flexibility of the linear strut and the self-expanding scaffold as a whole. The flexibility-increasing geometry may be in middle of the linear strut or positioned nearer to the proximal end of the linear strut (near the elongated tubular body) or nearer to the distal end of the linear strut, close to the attached sinusoidal ring, if applicable. The strut width of the flexibility-increasing geometry portion of the linear strut may be the same as that of the straight sections of the linear strut or they may be thinner. In the preferred example, the strut width of the flexibility-increasing geometry portion of the linear strut is approximately half that of the straight sections of the linear strut.

### Effect of Self-Expanding Scaffold Geometry

The combination of the length, diameter, and contour of the self-expanding scaffold is important in determining the delivery, expansion, aspiration, and re-collapse (if applicable) performance of the device. Since the expandable scaffold portion of the device is typically stiffer than any guidewire and/or adjacent device components, the length of the expandable scaffold may impact deliverability. Shorter scaffolds can articulate more easily through a tortuous vessel than longer scaffolds. Shorter lengths are also better suited to resisting collapse during aspiration, since during aspiration the applied vacuum results in a pressure differential between the ambient blood pressure on the outside of the scaffold and the lower blood pressure under vacuum on the inside of the scaffold. This pressure differential seeks to recollapse the scaffold back into the crimped state. Shorter lengths provide for both less total force applied to the scaffold (less area for the pressure to act upon) and for a shorter lever arm against which that force is applied. However shorter scaffolds have to expand wider in order to contact the vessel wall for proper sealing and aspiration, which may decrease clot aspiration efficiency. The width of expansion can be characterized by the “included angle” of the expanded scaffold.

FIG. 23 shows an example of conical scaffold 230 formed with a tapered serpentine body attached to a base ring which expands radially outwardly at an angle 231 in a distal direction. While an included angle of 180° (where the scaffold has expanded into a disk perpendicular to the axis of the catheter) would functional and feasible as it will seal the vessel and perform superior to a conventional aspiration catheter, such a configuration may not funnel clots into the aspiration lumen as well as a design with a more tapered entry would. Preferably, the self-expanding scaffold has an included angle between 20° and 120°, more preferably between 30° and 90°, and most preferably between 40° and 60°, in order to provide the best balance of deliverability and clot aspiration while maintaining sufficient vacuum resistance to avoid collapse. In a preferred example, the expandable scaffold is 2-3 mm long in the crimped state and expands to 4-5 mm diameter when unconstrained, which depending on the inner diameter of the aspiration lumen at the proximal end of the scaffold yields an included angle in the expanded state between 40° and 60°.

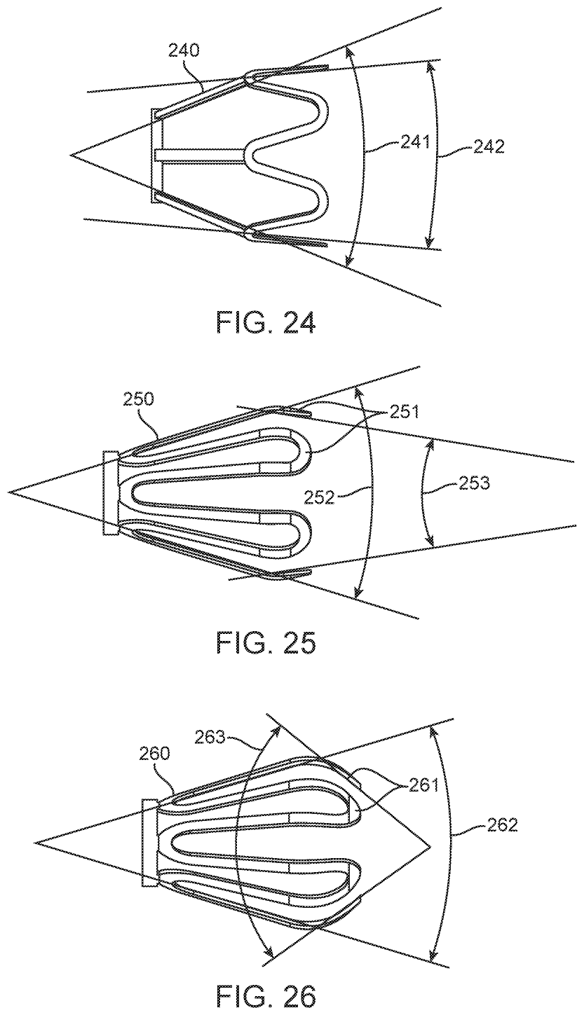

Some scaffold contours result in more than one angle within the scaffold which may result in a gentler and less potentially traumatic contact with the vessel and/or positively impact aspiration efficiency. Typically the distal portion of the scaffold will have a shallower angle while the proximal portion of the scaffold would have a steeper angle. FIG. 24 shows an example of conical scaffold 240 with a first and steeper proximal region included angle 241 and a second and shallower distal region included angle 242.

If the scaffold has been manufactured in a hemispherical or similar curved shape, the angle will increase smoothly along the length of the scaffold. In another example of the invention, the distal end of the scaffold has a reverse angle and in the expanded state the tips point into the lumen, such that if the expanded scaffold is advanced in the lumen the tip of the scaffold will help guide it along the vessel. FIG. 25 shows an example of conical scaffold 250 with inward-pointing tips 251, resulting in proximal scaffold region with included angle 252 and a distal scaffold region with reverse included angle 253. FIG. 26 shows a variant of the above example in which the scaffold 260 has more gradually curved and significantly inward-pointing tips 261, resulting in proximal scaffold region with included angle 262 and a distal scaffold region with steeper reverse included angle 263.

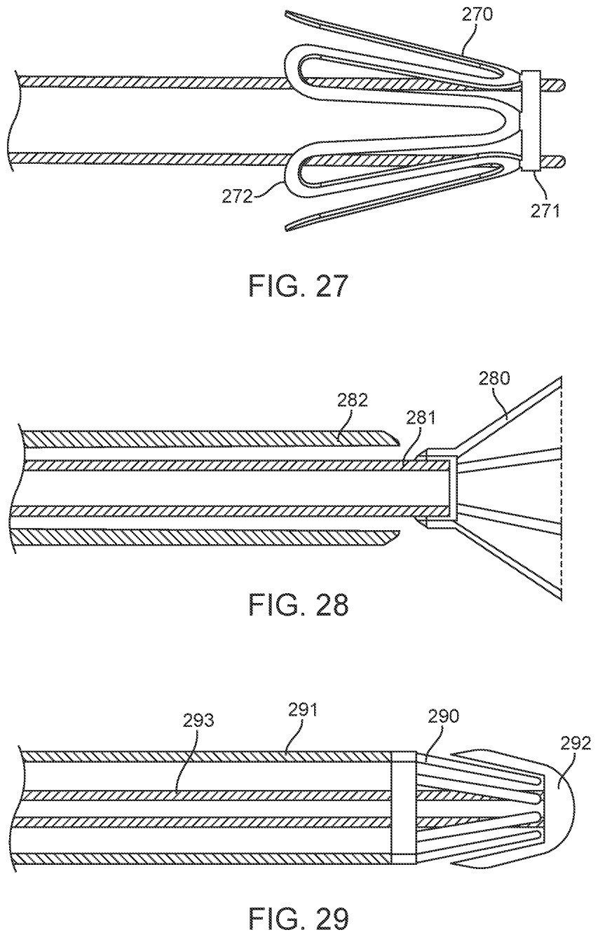

FIG. 27 shows another example of the present invention in which the self-expanding scaffold 270 is mounted with the apical end of the scaffold 271 distal and largest expanded diameter end 272 proximal. This example may utilize any of the self-expanding scaffold designs described elsewhere herein, as well as most of the constraint techniques discussed below. One advantage to using a reversed scaffold of this sort is that during aspiration the pressure gradient between the ambient blood pressure proximal to the expanded scaffold and the vacuum region distal to it seek to further open the scaffold and press it into the vessel wall, thereby providing a superior seal between the device and the vessel and maximizing vacuum levels and aspiration efficiency. Another advantage is that the device can be easily advanced deeper into the vessel, even during aspiration, in order to press into the clot or to capture more distal fragments not initially aspirated. In the preferred example, the system uses a drawstring design to facilitate collapse of the expanded umbrella after aspiration and prior to withdrawal from the anatomy.

### Means of Constraint and Release for Self-Expanding Scaffold

There are multiple means in which a self-expanding scaffold may be constrained during delivery through the vasculature to the site of treatment and thereafter expanded, and in some cases optionally may be collapsed after the aspiration treatment is complete.

FIG. 28 shows a preferred example in which the device comprises the self-expanding scaffold 280 attached to an inner elongated tubular body 281 which lay inside an outer elongated tubular body 282, with sufficient clearance between the inner and outer elongated tubular bodies to allow one to move distal and/or proximal with respect to the other. As manufactured and during delivery through the vasculature to the clot, the scaffold is kept sheathed in the collapsed state by the outer elongated tubular body. In this example the two tubular bodies are advanced together to the site of treatment, then the outer body is moved proximal with respect to the inner body, or the inner body is moved distal with respect to the outer body, thereby uncovering the self-expanding scaffold and allowing it to expand to the larger configuration for treatment. Alternatively, the physician may opt to advance the outer tubular body separately from the inner tubular body if such may provide a deliverability or other benefit, and then advance the inner tubular body with self-expanding scaffold within the outer tubular body as a secondary step.

In a preferred example, the outer tubular body has sufficient axial rigidity to allow it to be pulled back with respect to the inner tubular body to allow the self-expanding scaffold to expand, as well as be again advanced to close the self-expanding scaffold after aspiration. In another example the outer tubular body is intended only to be used in tension, which allows the outer tubular body to be pulled back and to release the self-expanding scaffold to expand, but not in compression in which the sheath requires sufficient compressive strength and buckling resistance to allow it to be advanced to re-collapse the self-expanding scaffold upon completion of aspiration. This example may be preferable when minimum profile and/or maximum aspiration lumen size is more desirable than the ability to return the self-expanding scaffold to the crimped state after aspiration is complete. The portion of the outer tubular body over the catheter intermediate segment and/or proximal segment may be drilled, notched, slotted, or otherwise cut to increase flexibility without significantly compromising tensile strength and stiffness.

In an alternate example, the constraining sheath only covers the scaffold, and possibly part of the catheter shafts, and is manipulated used a wire or catheter running through the aspiration lumen of the device and which is attached to the sheath. The wire or catheter may exit the distal end of the aspiration lumen through the scaffold distal tip, or through a port made for the purpose in the side of the device outer member.

FIG. 29 shows a preferred example in which the self-expanding scaffold 290 is attached to the distal end of the outer elongated tubular body 291 and is kept in the constrained state by a distal cap 292 attached to a removable inner elongated tubular body 293. In this example the two elongated tubular bodies are advanced together to the site of treatment, then the outer elongated tubular body is moved proximal with respect to the inner elongated tubular body, or the inner elongated tubular body is moved distal with respect to the outer elongated tubular body, thereby uncovering the self-expanding scaffold and allowing it to expand to the larger configuration. The inner elongated tubular body with distal cap is then withdrawn through the lumen of the outer elongated tubular body and removed from the device and patient's body, thereby allowing an unoccluded aspiration lumen.

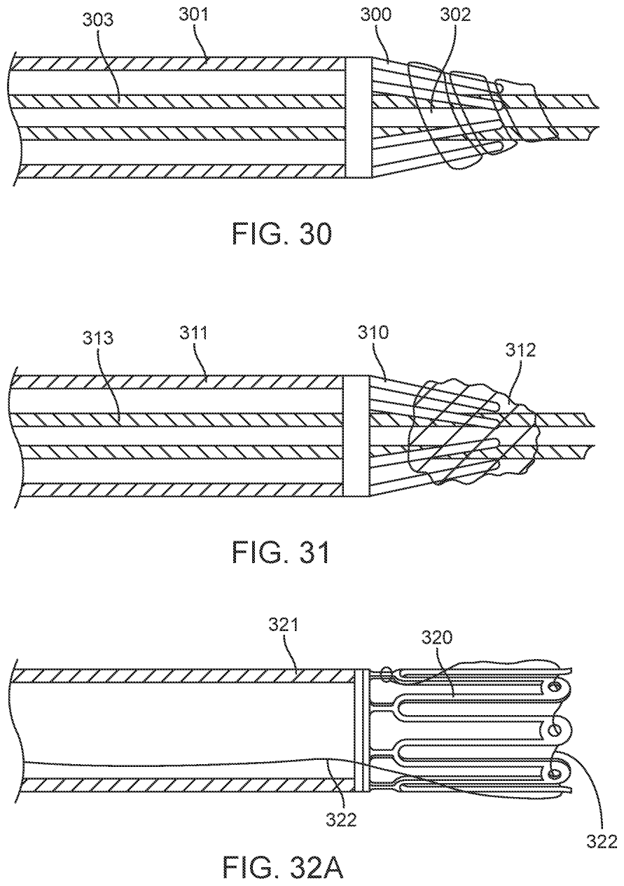

FIG. 30 shows a preferred example in which the self-expanding scaffold 300 is attached to the distal end of the outer elongated tubular body 301 and is kept in the constrained state by a wire, filament, or ribbon 302 wrapped around the at least distal end of the self-expanding scaffold and attached to a removable inner elongated tubular body 303. The wire, filament, or ribbon wraps over itself over the self-expanding scaffold thereby securing the end of the wire, filament, or ribbon not attached to the removable inner elongated tubular body, yet distal tension on the wire, filament, or ribbon causes it to unwrap easily and come free of the self-expanding scaffold. In one example, the wire is made from stainless steel, nitinol, a cobalt chrome alloy, titanium, or other metal of sufficient tensile strength and biocompatibility. In another example, the filament is made from nylon, PTFE, FEP, ePTFE, a suture material, or other polymer of sufficient tensile strength and biocompatibility. In another example, the wire or filament is of a substantially flattened cross-section such that the material resembles a ribbon more than a rod. In this example featuring a constraining wrapped wire, filament, or ribbon, the two elongated tubular bodies are advanced together to the site of treatment, then the outer elongated tubular body is moved proximal with respect to the inner elongated tubular body, or the inner elongated tubular body is moved distal with respect to the outer elongated tubular body, thereby unwrapping the wire, filament or ribbon from the self-expanding scaffold and allowing it to expand to the larger configuration. The inner elongated tubular body with the wire, filament, or ribbon is then withdrawn through the lumen of the outer elongated tubular body and removed from the device and patient's body, thereby allowing an unoccluded aspiration lumen. A relative advantage of this example versus the example featuring a cap is that as wrapped the wire, filament, or ribbon adds minimal stiffness to the system, and also once unwrapped can be easily withdrawn through the self-expanding scaffold and catheter shafts.

FIG. 31 shows an example in which the self-expanding scaffold 310 is attached to the distal end of the outer elongated tubular body 311 and is kept in the constrained state by a frangible material 312 which seals the at least distal end of the self-expanding scaffold to the removable inner member 313. The frangible material may be a water-soluble solid like a sodium chloride or potassium chloride salt crystal, a biodegradable polymer such as PLLA, or an adhesive gel. It may also be a solid scaffold made from polymer or metal which is securely attached to the removable inner member and which has loops or tabs covering the struts of the self-expanding scaffold to constrain it which can be broken away to release it. The two elongated tubular bodies are advanced together to the site of treatment, then the outer elongated tubular body is moved proximal with respect to the inner elongated tubular body, or the inner elongated tubular body is moved distal with respect to the outer elongated tubular body, thereby causing the self-expanding scaffold to break free of the frangible material and allowing it to expand to the larger configuration. The inner elongated tubular body and any remaining frangible material is then withdrawn through the lumen of the outer elongated tubular body and removed from the device and patient's body, thereby allowing an unoccluded aspiration lumen.

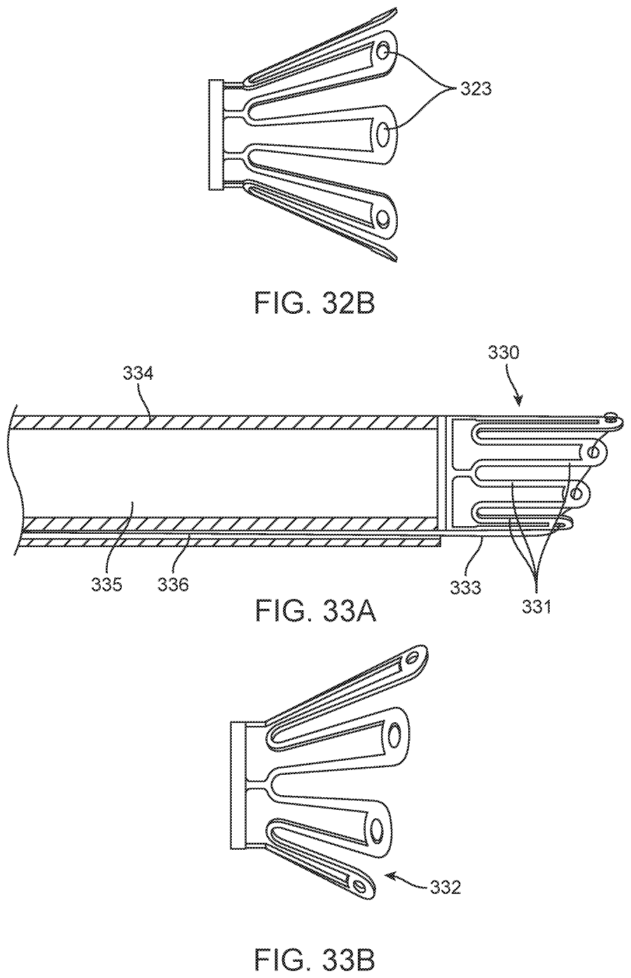

FIGS. 32A and 32B show another preferred example of the present invention in which the self-expanding scaffold 320 is attached to the distal end of an elongated tubular body 321 and is kept in the constrained state by a drawstring filament 322. FIG. 32B shows a closeup of the self-expanding scaffold featuring circular holes 323 at the distal end of the scaffold through which the filament is threaded. Pulling tension on the filament draws the arms of the scaffold together to reduce it to the collapsed state, while releasing the tension allows the self-expanding scaffold to reopen. In another example of the present invention featuring a filament, the self-expanding scaffold is initially constrained by another method of constraint described herein and the filament is used primarily to allow recollapse of the scaffold after release and expansion. This may allow for a tighter initially collapsed profile, and also for easier expansion because scaffold deployment is not hindered by friction of the filament sliding through the features.

Instead of holes the self-expanding scaffold may contain features such as slots, loops, rings or hooks instead of circular holes through which the filament is threaded, or the filament may be wound directly around the struts, crowns, or other struts in the self-expanding scaffold. In an alternative example, a second filament may wrap around the perimeter of the self-expanding scaffold and protrude through features in the scaffold like those described above or between natural gaps in the scaffold pattern, and the primary filament only laces through and pulls on the perimeter filament. An advantage to this approach is that filament does not need to be threaded directly through multiple struts of the scaffold, and/or it interfaces only with the perimeter filament, resulting in less friction in the assembly and smoother/easier operation.

In one example the filament runs the length of the catheter body to a slider or other mechanism on the handle which allows the physician to put it in tension or release said tension, thereby expanding or collapsing the scaffold. In another example the filament attaches to a wire, tube, or other component with torsional rigidity which runs the length of the catheter body, and this torsion component is rotated to wind or unwind the filament around it thereby pulling tension on it or releasing such tension. An advantage of using such a torque element arrangement is it omits any stretch in the filament being tensioned along the length of the shaft, and also eliminates any tendency of the filament tension causing the shaft to deflect.

The filament may be made from polymeric materials such as nylon, PEEK, FEP, PTFE, ePTFE, or UHMWPE filaments or ribbons, metals such as stainless steel, NiTi, cobalt chrome alloys, or titanium wires or ribbons, or any material providing similarly sufficient tensile strength and biocompatibility. The filament may be made from two or more components, for example with stiffer and more axially rigid components running along the proximal portions of the elongated tubular bodies, and more flexible and/or lower friction materials used in the more distal portions of the device. The filament may run inside the aspiration lumen of the device, in a separate channel substantially within the wall of the elongated tubular body, and/or immediately outside of the elongated tubular body in an attached channel.

If the design uses a torque element to wind or unwind the filament, the construction of such torque element would be as has been previously described for an inner torque member used for a coil distal segment design, except that in this case the torque element may run fully or partially outside the aspiration lumen, either free floating or in its own channel in either case.

FIGS. 33A and 33B show a variant of the above example in which the self-expanding scaffold 330 features struts 331 of different lengths, thereby reducing the angle 332 at which the filament 333 engages the first contact positions in the self-expanding scaffold and reducing friction of operation. In another example, two or more filaments are used to reduce the amount of contact points of each filament and friction of operation. FIG. 33A also depicts the use of a multi-lumen catheter shaft 334 with one dedicated aspiration lumen 335 and one dedicated filament lumen 336.

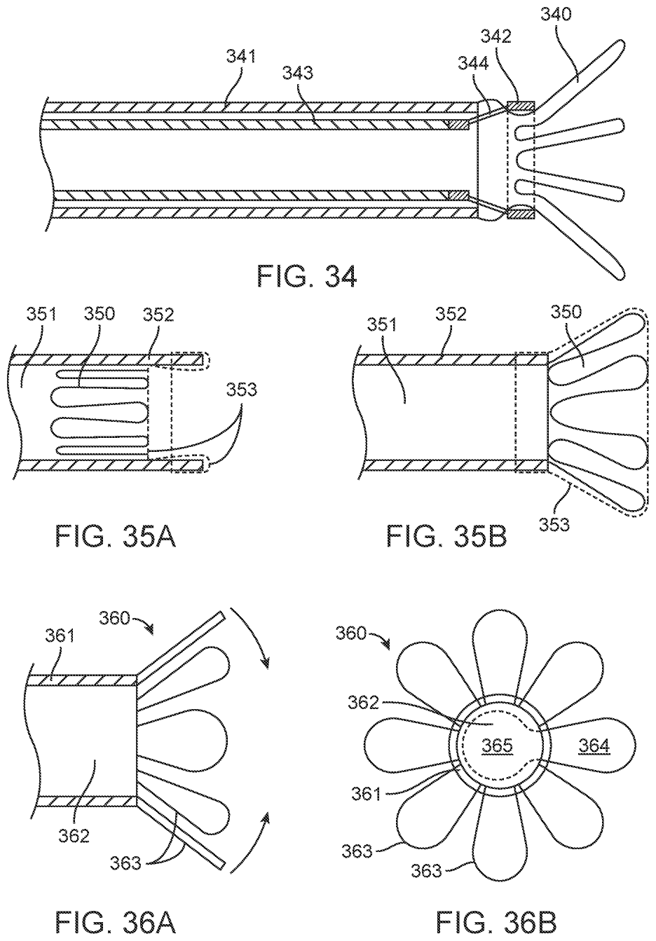

FIG. 34 shows another example of the present invention in which the self-expanding scaffold 340 is attached to the distal end of an elongated tubular body 341 and is kept in the constrained state by a ring 342 on the outside of the self-expanding scaffold, and the two are designed such that the ring can slide partially or completely along the self-expanding scaffold, such that when the ring is in a more distal position, the self-expanding scaffold is kept in the collapsed state, and when the ring is in a more proximal position the self-expanding scaffold is able to expand. The ring may be made of metallic, polymeric, or other material. Its position on the self-expanding scaffold is controlled from the proximal end of the device by wires, rods, or a tubular inner member 343 which extends continuously to the proximal end of the device. The ring may be corrected directly to the control wires, rod, or tubular inner member, or be part of a structure which includes for example links 344 connecting the constraint ring 342 to the inner member. The method of proximal control, whether wire(s), rod(s), and/or an elongated tubular member, may be positioned inside of or outside of the elongated tubular member to which the self-expanding strut is attached. In the preferred example, the constraining ring is laser cut from a nickel-titanium alloy and incorporates struts connecting it to a second ring bonded to an elongated inner member riding inside the outer elongated tubular member to which the self-expanding strut is attached. One key advantage to this design is that upon completion of aspiration, the constraining ring can be advanced to re-collapse the self-expanding strut for minimum vessel trauma during withdrawal from the patient.

FIGS. 35A and 35B show an example of the present invention in which the self-expanding scaffold 350 is capable of being compressed and folded into the fixed-diameter aspiration lumen 351 of the distal end of the catheter shaft 352, where it is retained by friction against the lumen or other components. The expandable segment is deployed by pushing it free of the lumen using a plunger wire or tube inside the aspiration lumen for this purpose, or the inner member can be used. In the example shown, the self-expanding scaffold comprises a sinusoidal ring scaffold which is only attached to the rest of the device at its distal end by the overlying vacuum resistant membrane 353, such that the sinusoidal ring scaffold can be crimped into a smaller cylindrical shape and inserted inside the catheter shaft 352, essentially turning the sleeve inside out. This example has the advantage that the self-expanding sinusoidal ring scaffold will continue to press outwards, keeping it firmly in position inside the catheter shaft while also maintaining a substantially clear lumen for passage of guidewire, microcatheters, and the like.

FIGS. 36A and 36B shows another example of the concept in which the scaffold is constrained by the aspiration lumen. In this example the self-expanding scaffold 360 is attached to the outer catheter shaft 361 which surrounds the aspiration lumen 362. The scaffold comprises loop struts 363 of outer perimeter slightly less that of the aspiration lumen 362, which are pressed into a circular shape and then folded across and slightly into the aspiration lumen like the petals of a flower. In the expanded state each loop is in the position 364, and when folded each loop is in the position 365. After being folded inside the aspiration lumen the loops will seek to return to a less circular state, thereby pressing against the inside of the aspiration lumen and remaining in the collapsed state until pressed free by a plunger wire, tube, or inner member in the aspiration lumen.

In another example of the present invention, the self-expanding scaffold is attached to the distal end of the outer elongated tubular body and is kept in the constrained state by features on the self-expanding scaffold such as struts, tines, hooks, linear or curved struts, flares, or other physical additions or alterations to the scaffold, which are themselves constrained from the inside of the self-expanding scaffold, thereby holding the entire self-expanding scaffold in the constrained state. In a preferred example the constraint-enabling features consist of linear struts attached to the distal end of the self-expanding scaffold which are then captured within an inner elongated tubular body. In this example the two elongated tubular bodies are advanced together to the site of treatment, then the outer elongated tubular body is moved distal with respect to the inner elongated tubular body, or the inner elongated tubular body is moved proximal with respect to the outer elongated tubular body, thereby releasing the linear struts and allowing the self-expanding scaffold to expand to the larger configuration. The inner elongated tubular body is then withdrawn through the lumen of the outer elongated tubular body and removed from the device and patient's body, thereby allowing an non-occluded aspiration lumen. In another example, the linear struts are of different lengths to aid in assembly of the device.

In another example of the present invention, the self-expanding scaffold is attached to the distal end of the outer elongated tubular body and is kept in the constrained state by capture features on the self-expanding scaffold such as holes, loops, or curves in the linear struts or sinusoidal ring which interface with a complementary geometry on the elongated inner member, thereby holding the entire self-expanding scaffold in the constrained state. In a preferred example the capture features consist of loops within the design of the self-expanding scaffold, and the complementary geometry is a crown-shaped structure bonded to or cut into the inner elongated tubular body. When the self-expanding scaffold is in the collapsed state, the peaks of the crown-shaped structure hook the loops within the self-expanding scaffold, thereby holding the system in the collapsed state. In this example the two elongated tubular bodies are advanced together to the site of treatment, then the outer elongated tubular body is moved distal with respect to the inner elongated tubular body, or the inner elongated tubular body is moved proximal with respect to the outer elongated tubular body, thereby disconnecting the crown-shaped geometry at the distal end of the elongated tubular inner member from the self-expanding scaffold such that it can expand to the larger configuration. The inner elongated tubular body is then withdrawn through the lumen of the outer elongated tubular body and removed from the device and patient's body, providing an non-occluded aspiration lumen. In another example, the self-expanding scaffold is held in the constrained state by one or more wires or hooked or curved rods attached to the inner member which are hooked into or looped through the capture features in the self-expanding scaffold. Alternatively, the elongated tubular inner member can be omitted and the capturing crown-shaped structure, wires, hooked or curved rods, or other means of capture extend directly to the proximal end of the device such that it can be manipulated by the user to release the constraint on the self-expanding scaffold and allow it to deploy.

The elongated tubular member(s) which may be used to constrain and deploy self-expanding distal scaffolds are manufactured from a cylindrical polymeric tube. The tube can be manufactured from nylon, Pebax, polyurethane, silicone, polyethylene, PET, PTFE, FEP, PEEK, polyimide, or other materials. Single wall thickness of the tubes would be between 0.001″ and 0.020″, preferably between 0.002″ and 0.010″, and most preferably between 0.003″ and 0.008″. Material hardness of the polymeric tube components would be between 50 A and 80 D. The elongated tubular member(s) can be constructed from a single polymer extrusion, or be assembled from multiple pieces of varying wall thicknesses and stiffnesses bonded together. The multiple pieces could be bonded together using adhesives, laser, RF, ultrasonic, or hot air heat bonds, be melted in an oven to merge with each other, or using other methods widely known in the industry. Any elongated tubular member(s) may be reinforced by coils and/or braids of metals or polymers to improve mechanical properties, in particular axial stiffness to provide for efficient push force transmission to the device tip in order to release a constrained self-expanding scaffold. Such reinforcing materials may include but are not limited to various alloys of stainless steel, cobalt chrome, nickel-titanium, platinum and platinum-iridium, PEEK, polyimide, Kevlar, and UHMWPE. Any coil may be a spring guide in which the adjacent loops of the coil are in direct contact with each other in order to provide maximum axial stiffness, shaft push, collapse resistance, and radiopacity. In an example of the present invention in which the self-expanding scaffold is laser cut from a tube, an additional portion of said tube not used for the self-expanding scaffold can be cut into a non-expanding coil, ring, spine, braid, and/or other geometry to aid in attaching the self-expanding scaffold to the adjacent catheter shaft, and/or to reinforce or provide the foundation for construction of such shaft. In particular a design with an axial spine provides for improved axial stiffness and push and pull force transmission along the length of the device.

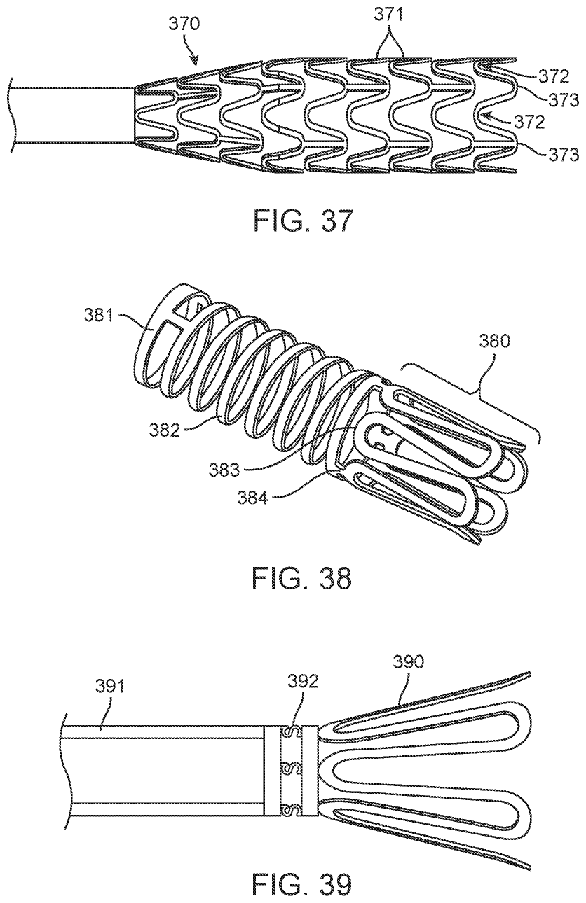

In another example of the present invention, the distal expandable segment only seeks to expand when exposed to moisture and/or heat. Exposure to such conditions causes the struts within the expandable scaffold to swell in width and/or length which due to the design of the scaffold thereby causes the entire scaffold to open. A slotted tube or sinusoidal ring type scaffold would be most suitable for this sort of design. FIG. 37 shows an example of a scaffold 370 composed of sinusoidal rings 371 made from a polymer which swells when exposed to moisture. When introduced into the body in the crimped state, the moisture in the patient's blood is pulled into the polymer, increasing stress on the inside of the folded crowns 372 more than it increases the stress on the outside of the folded crowns 373, thereby causing the crowns to unfold and the scaffold to expand. In this sort of design, the expandable distal segment may be constrained by any of the features, methods, or techniques describe herein for constraining a self-expanding scaffold, or the expandable distal segment may remain unconstrained and the device designed to expand at the appropriate rate in vivo.

Polymers examples suitable for use as a self-expanding scaffold which swell when exposed to moisture include graft polymers, block polymers, polymers with special functional groups or end groups such as acidic or hydrophilic type, or blend of two or more polymers. Polymeric material examples comprise one or more of Poly(lactide-co-caprolactone), Poly(L-lactide-co-ε-caprolactone), Poly(L/D-lactide-co-ε-caprolactone), Poly(D-lactide-co-ε-caprolactone), poly(glycolic acid), poly(lactide-co-glycolide, polydioxanone, poly(trimethyl carbonate), polyglycolide, poly(L-lactic acid-co-trimethylene carbonate), poly(L/D-lactic acid-co-trimethylene carbonate), poly(L/DL-lactic acid-co-trimethylene carbonate), poly(caprolactone-co-trimethylene carbonate), poly(glycolic acid-co-trimethylene carbonate), poly(glycolic acid-co-trimethylene carbonate-co-dioxanone), or blends, copolymers, or combination thereof. The polymeric material in this invention can be blends of two or more homopolymers such as polylactide, poly(L-lactide), poly(D-lactide), poly(L/D lactide) blended with poly(caprolactone), polyglycolide, polydioxanone, poly(trimethyl carbonate), or the like.

Polymers suitable for use as a self-expanding scaffold which change shape when heated to body temperature include poly(methacrylates), polyacrylate, polyurethanes, and blends of polyurethane and polyvinylchloride, t-butylacrylate-co-poly(ethyleneglycol) dimethacrylate (tBA-co-PEGDMA) polymers, combination thereof, or the like. These polymers exhibit shape memory properties and undergo a phase transformation at body temperature and seek to return to a pre-established state.

In another example of the present invention in which the distal expandable segment expands when exposed to moisture and/or heat, only part of the scaffold is composed of materials or struts sensitive to such stimuli, but which act on other non-sensitive parts within the scaffold to induce the entire scaffold to open.

In another example of the present invention, the distal expandable segment only expands when charged with an electric current. Upon application of the current the elements within the expandable structure seek to either shorten or lengthen which due to the design of the structure thereby causes the entire structure to open.

### Methods of Distal-Segment Attachment

The means by which a distal expandable segment is attached to the elongated tubular body of the intermediate segment can significantly impact the performance of the device with respect to profile, flexibility, deliverability, and aspiration, especially with a self-expanding scaffold design which tends to be stiffer than a coil design. In the simplest configuration, the distal expanding segment terminates proximally in a ring of approximately the same diameter as the adjacent shaft, and is intended to be butt-joined to the shaft or lap-joined inside or outside the shaft (see FIG. 32A for an example). The ring may be have a notch or slit allowing it to be stretched to crimp over the shaft or compressed to squeeze inside of it. While simple to manufacture and robust in tension or compression, this attachment approach may result in a locally stiff junction. A more flexible junction is desirable because it allows the distal portion of the device including the expanding structure to turn easily to follow the guidewire and track through vascular tortuosity. This aids ease of delivery to the site of treatment. Furthermore a flexible junction is desirable because when the distal segment is expanded, it will flex or swivel at the junction and self-align with the vessel. This aids vessel sealing and clot aspiration, especially in tortuosity.

FIG. 38 shows an example of a flexible junction design in which the distal expanding segment 380 is detached from the base attached to the catheter shaft 381 by a coil or pigtail structure 382. The coil or pigtail structure can flex easily improving the ability of the distal structure to conform to the vessel in both the crimped and expanded states, while an extension of the vacuum resistant membrane allows the system to maintain vacuum integrity. If axial movement in tension or compression is undesirable, and/or twisting needs to be resisted as part of an expanding coil design, the loops of the coil can be joined by links to restrict this without significantly impacting flexibility. If all such links are straight then it forms a spine-and-loops structure, or alternately one or more “M”, “S”, “U”, “W”, or other such curved links can be employed. Alternately or additionally, a polymer layer can be bonded over or into the pigtail to reinforce it against axial movement and/or provide for vacuum resistance. One or more of the proximal ends of the struts or crowns 383 of a self-expanding scaffold may be free floating and not connected to the pigtail structure except through adjacent struts or sinusoidal curves, which are either directly connected themselves or indirectly connected through links 384.

FIG. 39 shows another example of the present invention in which the distal expanding structure 390 is connected to the adjacent catheter shaft 391 using one or more “S”, “M”, “U”, “W”, or other such flexible links 392.

FIG. 40 shows another example of the present invention in which the distal expanding structure 400 is connected to the adjacent catheter shaft 401 using one or more ball-and-socket type joints 402. Such joints may be substantially 2 dimensional or 3 dimensional in nature.

FIG. 41 shows another example of the present invention in which the distal expanding structure 410 is entirely disconnected from the adjacent catheter shaft 411 except by the vacuum resistant membrane 412. The distal expanding structure may be a single uniform structure, or comprise multiple independent elements with free distal and/or proximal ends coupled only by the membrane.

### Alternate Designs and Mechanisms for the Distal Expandable Segment

In addition to the various coil and self-expanding scaffold designs previously described, there are several alternate means of creating a reversibly driven distal expandable segment utilizing a design which has a crimped/collapsing/opening or folding/unfolding structure which expands and collapses when acted upon by an mechanical force such as a pushrod, pull wire, torque shaft, or hydraulic force.

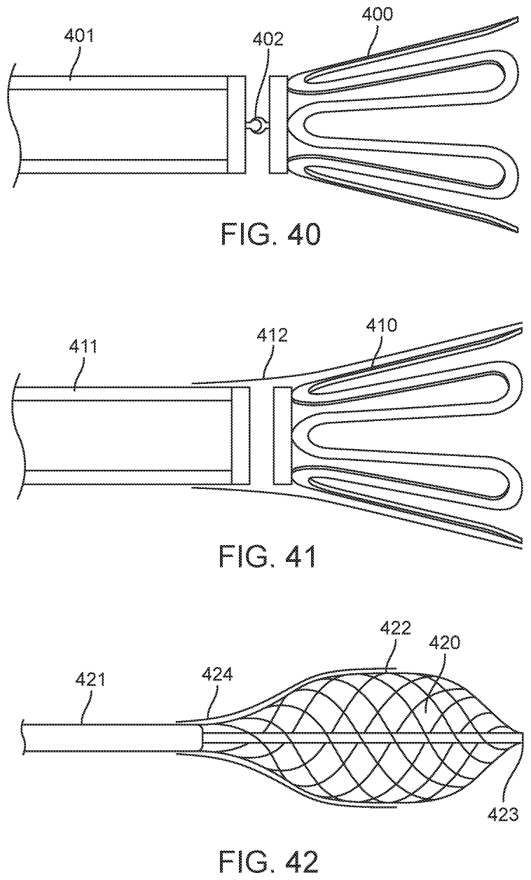

FIG. 42 shows an example of the present invention in which the distal expandable segment comprises a braided structure 420 which flares outwards from the catheter shaft 421 to the intended maximum expansion diameter 422 and then tapers down to connect to an inner member at its distal end 423. The inner member is torqued and/or pushed and pulled to open and close the distal expandable segment. The vacuum resistant membrane 424 covers the braided structure up to approximately its point of maximum diameter, while distal to that the structure is an open mesh through which the clot can be aspirated. In the preferred example, the braid uses thin wires and/or a smaller number of wires so as to provide the most open mesh possible and not obstruct clot aspiration, and/or is designed such that the braid wires splay during expansion of the distal segment thereby leaving distal areas with more concentrated wires and areas that are substantially open and more amenable to unobstructed clot aspiration.

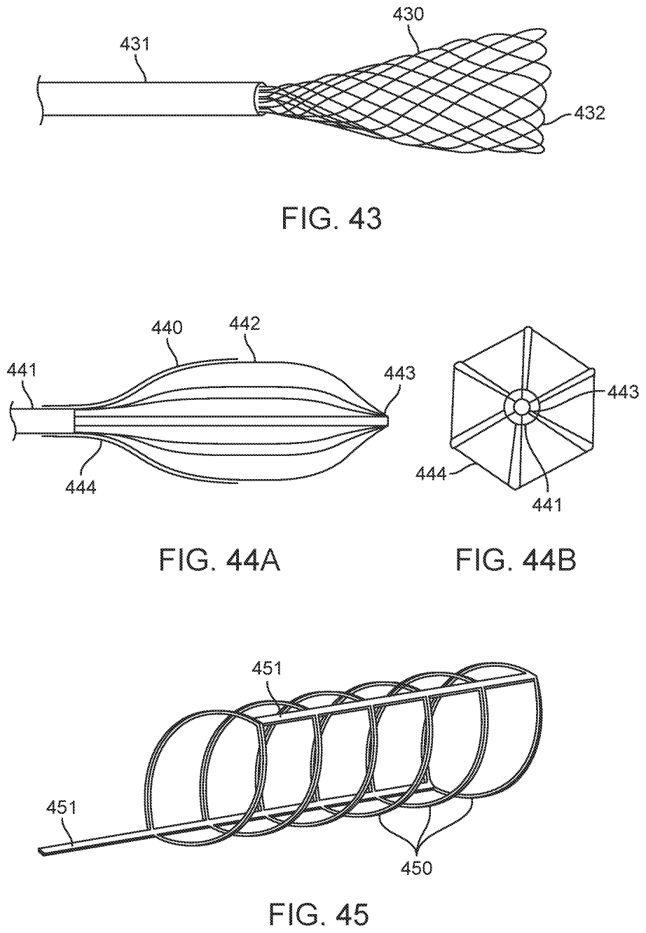

FIG. 43 shows an example of the present invention in which the distal expandable segment comprises a braided structure 430 attached to and flaring outward from the outer catheter shaft 431, which at its distal end 432 connects to a second inner braided structure attached to and flaring outward from an inner catheter shaft. In a manner of operation similar to that of the two coil system shown in FIGS. 12 and 13, the inner member and inner braid is rotated against the outer catheter and braid causing the two braids to push against each other and expand.

FIGS. 44A and 44B show an example of the present invention in which the distal expandable segment comprises a longitudinal ribbed structure 440 which flares outwards from the catheter shaft 441 to the intended maximum expansion diameter 442 and then tapers down to connect to an inner member at its distal end 443. When the inner member is pulled the ribs are put in compression which causes them to bow outwards thereby expanding their profile, and when the inner member is pushed the rib are put in tension which causes them to stretch flatter thereby contracting their profile. In a preferred variant of the present example, one or more V links or other means are used to attach the ribs to each other in order to maintain their circumferential alignment. The vacuum resistant membrane 444 covers the ribbed structure up to approximately its point of maximum diameter, while distal to that the structure the ribs are open through which the clot can be aspirated.

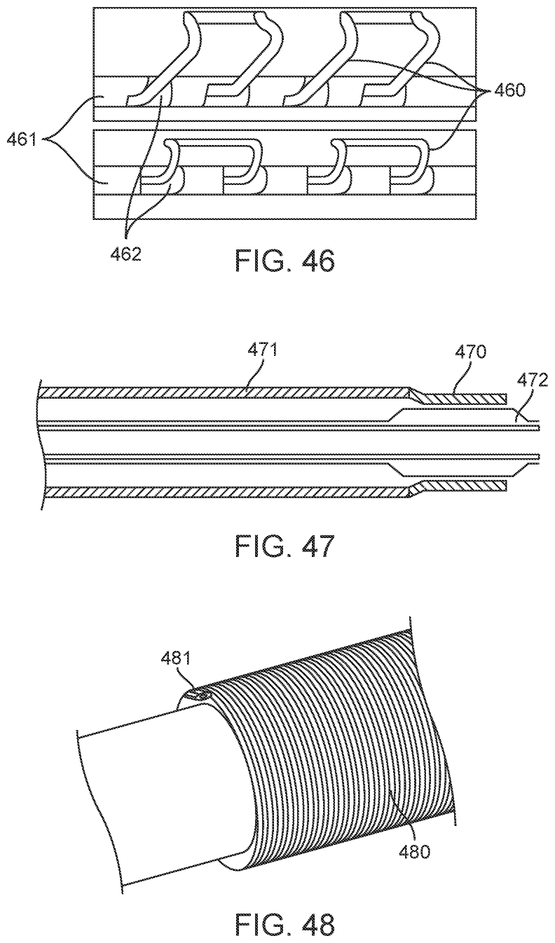

FIG. 45 shows an example of a distal expandable segment comprising multiple rings 450 connected on opposite sides to spines 451. One spine is attached to the catheter outer member of the intermediate segment, such that pushing or pulling the other spine causes the rings to fold open, thereby expanding the structure. The rings do not need to be circular and may be able to be further squeezed by a sheath or other constraint for minimize profile in the collapsed state. Optimally this sort of design would be laser cut from a NiTi tube in order to create a single robust but functional structure.

FIG. 46 illustrates a variant of the above example a structure consisting of rings 460 and a single spine has the spine covered by a tubular structure 461 with cuts 462, such that as the spine is pulled proximal into the tubular structure, the rings of the expandable distal segment are forced into a collapsed position by cuts in the tubular structure, and likewise when the spine is advanced the rings become unconstrained and expand.

FIG. 47 shows another example of the present invention in which the distal expandable segment comprises a slotted tube, sinusoidal ring, spine with ribs, or other plastically deformable scaffold 470 mounted to the end of an outer elongated tubular body 471, inside of which is a balloon catheter 472 which is inflated to expand the scaffold. After the scaffold has been expanded the balloon catheter is deflated and is then withdrawn through the lumen of the outer elongated tubular body and removed from the device and patient's body, thereby allowing an non-occluded aspiration lumen.

FIG. 48 shows another example of the present invention in which the distal expandable segment is constructed from a coiled polymer tubing 480 wherein the coil loops are bonded together. Pressurization of the lumen 481 within the polymer tubing from which the coil is constructed causes the material to elastically and/or plastically stretch and/or unfold any folds in the material, thereby causing the distal expandable element to expand or to unfold from a crimped configuration. Removal of such pressure causes the material to relax back to an at least partially collapsed state.

### Vacuum Resistant Membrane

In order to ensure integrity of the vacuum lumen over the distal expandable segment, a vacuum resistant membrane is attached to the scaffold. The membrane may lay on top of the scaffold, be bonded to the inner surface, or coated over the scaffold such that it forms a film between the ribbons and struts of the structure. In a preferred example the membrane is attached to the intermediate segment proximal to the scaffold, and may also be attached to elements of the scaffold at one or more points or be free to move independently of them. In an alternate example, the membrane is attached to at least the distal portion of the scaffold. As the scaffold expands, the membrane stretches or unfolds with it, approximately matching the diameter of the scaffold. In designs involving a coil distal segment in which the coil is unwound to expand, the vacuum resistant membrane may cling to the coil and twist as the unit expands, potentially compromising expansion of the coil and functionality of the device. A key intent of the present invention is to disclose a number of techniques by which such membrane twisting can be mitigated or avoided.

For example, the membrane can be attached firmly only at the distal end of the coil such that it spins with the coil while the latter expands, and/or be anchored at the proximal end in a manner that allows the membrane to spin with respect to the shaft as the coil expands, yet not move proximal or distal. Typically such an arrangement involves two circumferential rings or ridges around the distal end of the catheter shaft, and a compatible ring or rib on the inside of the proximal end of the membrane which fits between the two. Alternately, a separate and more structurally robust element with such a ring or rib may be used to which the proximal end of the vacuum resistant membrane is then attached.

In another example, the vacuum resistant membrane comprises several independent pieces of material in a series of overlapping skirts, which are each attached to the coil and can rotate independently of each other, yet are pulled together under aspiration to provide a substantially vacuum-tight structure. In an extension of this concept, the vacuum resistant membrane may comprise a polymer ribbon bonded to the entire length of the coil ribbon, in which the polymer ribbon is sufficiently wider than the coil ribbon to overlap the adjacent coil loops in the expanded state, thereby providing a substantially vacuum-tight structure under aspiration.

There are many means of creating the vacuum resistant membrane. The membrane may be fully elastic, and fit snugly onto the scaffold in the collapsed state. As the scaffold expands the membrane stretches to accommodate the increased diameter, then when the scaffold is recollapsed the elastic membrane relaxes back to a small diameter.

Membranes may also be semi-elastic or non-elastic, and in their natural unstressed state be of a diameter larger than that of the fully collapsed scaffold, either similar to the vessel size or at a convenient intermediate dimension. The membrane is then twisted, wrapped, folded, furled, or otherwise reduced in profile to match the profile of the scaffold in the collapsed state to aid device deliverability. A heat set may be used to help keep membranes of this sort at a reduced profile, and/or a very thin elastic tube or bands may be placed over the folded membrane. Non-elastic membranes of this type simply unfold as the scaffold are expanded, then refold naturally as the scaffold are collapsed or remain loose and unobstructive around the collapsed scaffold. Typically the scaffold will be collapsed only after the clot has been extracted, in which case aspiration will be active and the vacuum will help refold the membrane.

Elastic membranes may be made from a variety of soft polymers in the silicone, polyurethane, and polyamide families. Examples included C-flex (silicone), fluorosilicone, Tecothane (polyurethane), and Pebax (polyamide). Some name brand polymers suitable for this application which generally fall into one or more of the above polymer families include Chronoflex, Chronoprene, and Polyblend. Membranes in the hardness range of Shore 50 A through 40 Durometer work best. At the upper end of this scale a portion of the membrane stretch is plastic, not elastic, but enough of it is elastic to fulfill the recovery needs.

Non-elastic membranes may be made from any of the materials used for the elastic membranes, just manufactured at a larger diameter, or from firmer materials in the 50-80 Durometer hardness range. Examples included various polyurethanes, Pebax 55 D, 63 D, 70 D, and 72 D, Nylon 12, PTFE, FEP, and HDPE. Thin metallic foils or foil-polymer laminates may also be used for a vacuum resistant membrane, providing a low friction and potentially radiopaque membrane. ePTFE (expanded polytetrafluoroethylene) is soft and flexible and makes an excellent vacuum resistant membrane, but is slightly porous which can compromise vacuum force application. An ePTFE membrane can be coated or covered with a thin layer of another material to eliminate the porosity. Typically this secondary material would be of the same materials and mechanical properties as those used for the elastic membranes described above. Other slightly porous meshes may find similar utility as a vacuum resistant membrane, with or without an additional porosity-eliminating layer.

In another example of the present invention, the vacuum resistant membrane may be made from a polymeric material which tends to absorb moisture and/or relax when warmed. Particularly useful for unfolding membrane designs, use of these materials may help the membrane to expand easily with the distal expandable segment. Such moisture and heat sensitive materials may also be coated over ePTFE or other membrane materials to promote the expansion of the latter, either as a continuous coated layer or in stripes or segments. Polymers suitable for use as a vacuum resistant membrane which swell when exposed to moisture include graft polymers, block polymers, polymers with special functional groups or end groups such as acidic or hydrophilic type, or blend of two or more of Poly(lactide-co-caprolactone), Poly(L-lactide-co-ε-caprolactone), Poly(L/D-lactide-co-ε-caprolactone), Poly(D-lactide-co-ε-caprolactone), poly(glycolic acid), poly(lactide-co-glycolide, polydioxanone, poly(trimethyl carbonate), polyglycolide, poly(L-lactic acid-co-trimethylene carbonate), poly(L/D-lactic acid-co-trimethylene carbonate), poly(L/DL-lactic acid-co-trimethylene carbonate), poly(caprolactone-co-trimethylene carbonate), poly(glycolic acid-co-trimethylene carbonate), poly(glycolic acid-co-trimethylene carbonate-co-dioxanone), or blends, copolymers, or combination thereof. The polymeric material in this invention can be blends of two or more homopolymers such as polylactide, poly(L-lactide), poly(D-lactide), poly(L/D lactide) blended with poly(caprolactone), polyglycolide, polydioxanone, poly(trimethyl carbonate), or the like. Polymers suitable for use as a vacuum resistant membrane which change shape when heated to body temperature include poly(methacrylates), polyacrylate, polyurethanes, and blends of polyurethane and polyvinylchloride, t-butylacrylate-co-poly(ethyleneglycol) dimethacrylate (tBA-co-PEGDMA) polymers, combination thereof, or the like. These polymers exhibit shape memory properties and undergo a phase transformation at body temperature and seek to return to a pre-established state.

The membranes may be extruded, dip coated on a mandrel, sprayed over a mandrel, electrospun, or manufactured using other means common in the industry. The membranes may be used “as is”, or further necked, stretched, or blow molded to achieve desired dimensions and properties. Wall thicknesses are ideally low to maintain a low device profile, ranging from 0.0005″ to 0.005″. The membranes may be configured in a cylindrical, tapered, reverse tapered, convex profile, concave profile, or other shape as preferred in order to expand smoothly and without twisting and perform as desired.

The membranes may be attached to the catheter shaft and the scaffold struts by any of the means in common use in the industry, including adhesives, heat shrink tubing entrapment, heat bonding, mechanically crimping under a swaged metal band, tying or riveting, etc.

The outside of the membrane may be coated with a lubricious coating to aid deliverability into the anatomy. In some cases the membrane may be inclined to twist as the coil or other rotating scaffolds in the distal segment are expanded or collapsed in profile. If the twisting is not desirable, the outside of the scaffolds and/or inside of the membrane may be lubricated to aid free movement of the scaffolds inside the membrane. Preferred lubricants include a hydrophilic coating of chemistry known in the industry, silicone oil, and PTFE spray coatings. The membrane can also be designed to incorporate wires or a braid to resist twisting.

Another example to mitigate or eliminate membrane twisting over the coil, to provide for a more circular distal end to the aspiration lumen, and to otherwise influence distal segment expansion dynamics is to place an expandable/collapsible structure between the coil scaffold and the membrane inside of which the coil scaffold can freely spin, such as a NiTi wire braid or PTFE slotted tube. More than one such structure may provide improved performance compared to a single structure. In a preferred example the expandable/collapsible structure, also referred to as the liner, is designed to resist twisting while at the same time requiring minimum force to expand. Suitable materials for this application include PTFE, FEP, HPDE, and other low friction polymers. Self-expanding materials such as nickel-titanium alloys and the various polymers which swell when exposed to moisture and/or change shape with heat (previously described) are also suitable for use as a liner, since their self-expansion force can be tuned to substantially counteract any compressive force exerted by an elastic vacuum resistant membrane, or to promote opening of a folding vacuum resistant membrane design. Such liners are typically laser cut from a tube into a slotted tube pattern, preferably with a spiral aspect to aid flexibility and while maintaining a continuous torque-resisting pattern. The liners can also be made from a polymer mesh or filter material with similar expandable properties. Liners may range in thickness from 0.0005″ to 0.008″, more preferably 0.001″ to 0.005″, and most preferably about 0.003″. The outside and/or inside of the liner may be coated with hydrophilic coating, silicone oil, PTFE spray, or other lubricant to aid in allowing the components to slide freely past each other during distal segment expansion and contraction. Alternately, one or more surfaces of the liner may be increased in roughness using sandpaper, microblasting, or other means in order to promoted adherence of one component to another where advantageous, for example helping the membrane to stick to the liner such that the combined structure is more resistant to twisting than the sum of the two individual parts.

In another example of the liner concept, the liner(s) are shorter than the membrane and positioned selectively. For example, a 2-3 mm long liner at the distal end of the membrane may aid membrane robustness during track and promoted a circular and collapse-resistant aspiration lumen. In another example, a liner in the middle of the distal expandable segment is used to selectively reinforce the membrane and promote or retard expansion in that area.

In an alternate example, one or more free-rolling wires are positioned between the coil and the vacuum resistant membrane and are used to prevent the membrane from clinging to the coil and twisting, in a manner akin to that of a needle bearing. Such wires will typically be in the range of 0.001″ to 0.005″ in diameter and may be made from stainless steel, cobalt chrome, nickel-titanium, polyimide rod, or any other sufficiently robust material.

In a further example of the present invention, the vacuum resistant membrane is attached to a sheath on the outside of the outermost elongated tubular member of the device, and the sheath extends from the proximal end of the vacuum resistant membrane to the proximal end of the catheter where it is integrated into the handle. This outer sheath is used to provide tension and/or counter-torque force to the vacuum resistant membrane during expansion of the distal segment to prevent membrane bunching or twisting. The portion of the sheath over the catheter intermediate segment and/or proximal segment may be drilled, notched, slotted, or otherwise cut to increase flexibility without significantly compromising tensile and/or rotational strength and stiffness.

It may also be advantageous for the vacuum resistant membrane to cover only part of the scaffold, such that scaffold extends distal to the distal end of the membrane.

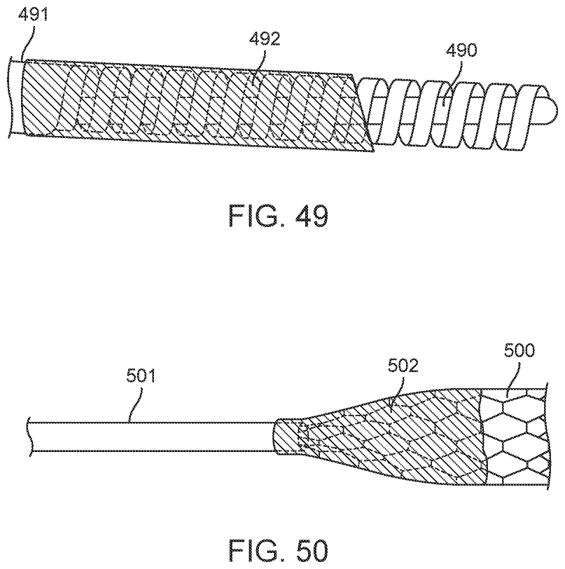

FIG. 49 shows an example of a distal expanding segment comprising a coil 490 attached to a catheter shaft 491 in which the vacuum resistant membrane 492 extends from the end of the catheter shaft to a point substantially proximal to the distal end of the coil.

FIG. 50 shows an example of a distal expanding segment comprising a self-expanding scaffold 500 attached to a catheter shaft 501 in which the vacuum resistant membrane 502 extends from the end of the catheter shaft to a point substantially proximal to the distal end of the self-expanding scaffold.

One potential advantage of a configuration in which the distal portion of the scaffold is not covered by the membrane is that the uncovered portion of the scaffold in its collapsed state can be used to penetrate the clot, such that when the scaffold is expanded it disrupts the clot aiding aspiration and removal from the body. The expanding scaffold may break up the clot as the ribbons or struts are forced through the clot, or it may stretch the clot into a ring such that when the device is withdrawn the clot is invaginated for better aspiration or otherwise well anchored to the scaffold assist the vacuum force in pulling out the clot intact. In one example of the design, the scaffold comprises features designed to assist in mechanically disrupting the clot during expansion, such as sharp edges, metallic protrusions, fins, hook elements, or slots which serve to improve cutting or gripping of the clot.

### Scaffolds Comprising Single, Continuous Element

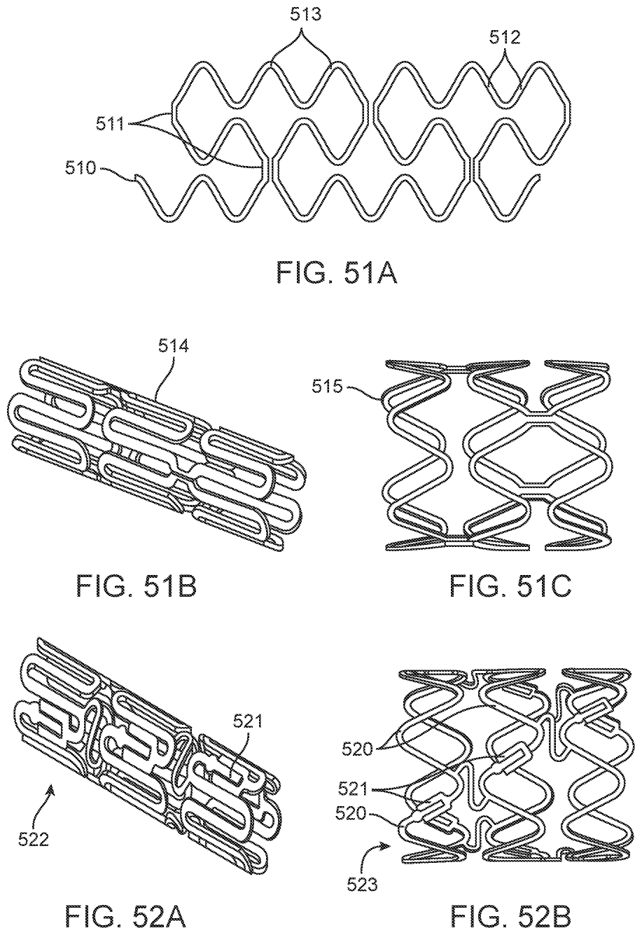

In another example of the present invention the distal expandable segment comprises a self-expanding scaffold of a generally sinusoidal or serpentine ring design, and the structure of the scaffold is provided by a single continuous undulating element or strut. FIG. 51A shows the pattern of the single undulating element 510 in a flattened state, as if the scaffold was bisected longitudinally and unrolled. The element contains longitudinally straight sections 511, angled sections 512, and curved sections or crowns 513. FIG. 51B shows the scaffold in the collapsed state 514 and FIG. 51C in the expanded state 515.

The primary advantage of this design is that the scaffold has superior flexibility in bending, tension, compression, and torsion compared to conventional sinusoidal ring scaffold designs with multiple continuous sinusoidal rings and/or multiple connection points within the pattern. The superior flexibility allows for easier delivery in tortuous anatomy, better conformance to the vessel in the expanded state, improved vessel sealing and less blood leakage during aspiration, and reduced vessel trauma. At the same time the scaffold of the present example maintains substantially equivalent radial strength and ability to support the vessel and resist vacuum compression as a conventional scaffold of similar material and dimensions.

FIGS. 52A and 52B show another example of the present invention in which the scaffold comprises multiple continuous undulating elements 520, which are not in continuity with each other but are maintained in position by tab-and-slot joints 521. FIG. 52A shows the scaffold in the collapsed state 522 and FIG. 52B in the expanded state 523. Alternately the joints may be of a ball-and-socket, hook and hole, male and female, nesting “S” curves, or other design which restrict movement of the multiple elements in at least one direction but allow movement in other directions, thereby granting the scaffold increased flexibility compared to a scaffold with metallic material continuity at the junctions. One or more joints may be bonded with a polymeric or elastomeric material configured to hold one or more of the multiple continuous undulating elements together during expansion and to thereafter form at least one discontinuity in the circumferential ring and the axial link after expansion of the scaffold in a physiologic environment. In the preferred example any such bonding material is a biodegradable polymer and/or adhesive.

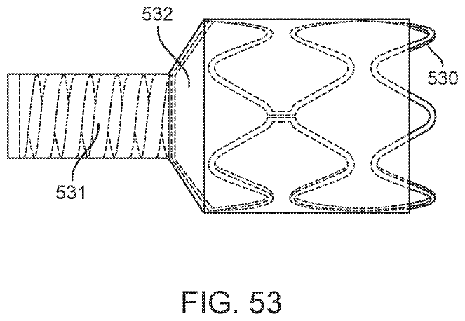

FIG. 53 shows an example of a distal expanding segment scaffold 530 featuring a single continuous undulating element as attached to the intermediate segment 531 of the aspiration catheter of the present invention and covered with the vacuum resistant membrane 532. The scaffold is shown in the expanded state after constraint removal. Compared to an aspiration catheter feature a self-expanding scaffold design with a conventional sinusoidal ring structure, the distal segment of the aspiration catheter of FIG. 53 would be more flexible during delivery and more conformable to the vessel wall after expansion, improving sealing with the vessel and minimizing leakage around the edge of the scaffold during aspiration.

In the preferred examples of the present invention featuring a scaffold with one or more continuous undulating elements, as depicted in FIGS. 51A-53, the scaffold is laser cut from a NiTi hypotube and after cleaning and polish is heat set to the desired configuration in the expanded state. After assembly onto the catheter shafts, the scaffold is then pressed into the collapsed state and constrained with a sheath, cap, or other means as previously described for self-expanding scaffolds. In an alternate example, the scaffold can be made from a material which self-expands when exposed to moisture, heat, and/or electricity such that a separate constraint is unnecessary. The strut widths and thicknesses, expansion diameters, straight and tapered profiles, catheter construction, and other features of the self-expanding scaffold are otherwise the same as has been previously described for self-expanding scaffold designs comprising sinusoidal rings.

In another example of the present invention featuring a scaffold with one or more continuous undulating elements, as depicted in FIGS. 51A-53, the scaffold is made from stainless steel, a cobalt chrome alloy, titanium, or other non-superelastic material and is expanded using a balloon as depicted in FIG. 47.

### Method of Manufacture and Assembly—Example 1 for Twin Coil Distal Expandable Segment

In the exemplary dual coil example, nickel-titanium hypotubes are laser cut to create the coils used in the distal expandable element. The coils are then chemically and/or mechanically de-slagged and then electro-polished. The electropolishing process smooths the surface of the coils and rounds the edges, causes the cross-section geometry of the ribbon to become more circular. The more circular cross-section has lower contact area between the outer and inner coils which reduces friction between the two and aids collapse and expansion.

The coils are then placed over a stainless steel rod or hypotube and heat treated in a fluidized temperature bath filled with aluminum oxide sand to set the desired neutral state. They are then removed from the bath and quenched in water. The heat treatment process allows the coils to accommodate greater diametric expansion due to the change in geometry.

The various catheter shafts are cut to length and heat bonded to each other using conventional means such as laser bonding or a hot air nozzle. If the materials are chemically incompatible then adhesives may be used. The catheter outer member constructed as follows. First a PTFE liner is stretched over a steel mandrel. Next the proximal portion of the lined mandrel (eventually forming the proximal shaft segment) is braided with a stainless steel braid. Then the distal portion of the lined mandrel is wound with a coil (eventually forming the intermediate shaft segment). Polymer sections of appropriate length and wall thickness are slid over the braided and coiled portions of the assembly, then the entire assembly covered with heat shrink tubing. The assembly is placed in an oven at 160 C for approximately 10 minutes to cause the polymer outer jacket to melt and flow around the braid and coil, thereby forming a robust cohesive structure after the heat shrink tubing is removed. The catheter inner member is formed in the same manner as described for the outer member above.

The outer coil is then bonded to the catheter outer tubular member using adhesive, a heat melt, overlying heat shrink, or other methods. Typically the proximal end of the outer coil will be designed with a slot or other gap allowing the hypotube stub to be crimped down to the desired diameter before bonding, and may have axially aligned legs to aid bonding. The coil may be bonded inside, outside, or in a butt joint with the adjacent shaft. Alternately, the component can be laser cut from a single piece in which one portion of the coil becomes the expandable distal segment and another portion of the coil is polymer jacketed and bonded to form the intermediate segment as described above, thereby saving the need for a separate distal segment to intermediate segment bond.

The inner coil is likewise bonded to a catheter inner tubular member which can rotate inside the outer tubular member. The inner coil assembly is threaded through the outer coil assembly until the distal end of the outer and inner coils align, then the coils are attached together using wires, tabs, or welds.

The proximal ends of the catheter outer and inner tubular members are trimmed to length and bonded to their receptive parts in the handle mechanism. The handle mechanism is then used to rotate the catheter inner tubular member concentrically within the outer tubular member such that the coil is collapsed to the desired size. At this point the vacuum-resistant membrane is slid over the coils and bonded to the distal end of the catheter shafts to form the complete expandable distal segment. If a non-elastic membrane is used, it may be heat set into the folded shape either before or after attachment to the device.

The portion of the device which will be in contact with the blood vessels will be coated with a hydrophilic coating or other lubricious coating to aid device delivery in vivo. A lubricious coating or material may also be applied to the inside surface of the scaffold and/or aspiration lumen of the catheter shafts in order to facilitate smooth movement of the device over guidewires and microcatheters, and to promote rapid clot aspiration. The completed device is then packaged and sterilized.

Construction of a single coil example is generally similar, except that there is only one coil and the catheter tubular inner member will extend to the tip of the single coil. Various alternative means of assembling the device of the present invention are envisioned. For example, the coils may be wrapped separately and secured in the fully collapsed state using special fixturing, the order of assembly may vary.

### Method of Manufacture and Assembly—Example 2 for Self-Expanding Scaffold

The self-expanding structure is laser cut from a tube made from a super-elastic nickel-titanium alloy, which is afterwards heat set into the desired expanded shape. In the preferred method, the expansion process is performed in multiple heat set steps using various mandrels with increasing diameters at each step.

The heat set scaffold is then electropolished to provide a smooth surface finish. The catheter shafts are constructed in the same manner as described for a coil design above. A short section of molded polymer sleeve is bonded to the distal end of the inner member. The scaffold is then bonded to the catheter shaft in the same manner as described for a coil design above. The vacuum resistant membrane is attached to the scaffold in the same manner as described for a coil design above.

The inner member is inserted through the outer member and scaffold.

A crimp fixture is used to press the scaffold and membrane to the collapsed state, whereupon the inner member is drawn back so that the collapsed scaffold and membrane is inserted into the polymer sleeve on the distal end of the inner member, thereby forming a constraining cap. The proximal ends of the catheter outer and inner tubular members are trimmed to length and bonded to their receptive parts in the handle mechanism. The portion of the device which will be in contact with the blood vessels will be coated with a hydrophilic coating or other lubricious coating to aid device delivery in vivo. A lubricious coating or material may also be applied to the inside surface of the scaffold and/or aspiration lumen of the catheter shafts in order to facilitate smooth movement of the device over guidewires and microcatheters, and to promote rapid clot aspiration. The completed device is then packaged and sterilized.

1. An aspiration catheter for removing clot from a blood vessel, said aspiration catheter comprising:
a catheter body having a proximal end, distal end, and an aspiration lumen therebetween;
a scaffold extending distally from the distal end of the catheter body and having a central clot-receiving passage contiguous with the aspiration lumen of the catheter body, wherein at least a portion of the scaffold is radially balloon expandable from a delivery configuration to an extraction configuration; and
a vacuum-resistant membrane covering at least a portion of the scaffold to establish a clot aspiration path from at least said portion of the scaffold to a proximal end of the aspiration lumen in the catheter body so that applying a vacuum to the proximal end of the aspiration lumen can draw clot into the central clot-receiving passage;
wherein at least some of the circumferential rings are circumferentially separable, joined by circumferentially separable axial links, and configured to circumferentially separate along separation interfaces.

2.-30. (canceled)

31. An aspiration catheter for removing clot from a blood vessel, said aspiration catheter comprising:
a catheter body having a proximal end, a distal end, and an aspiration lumen therebetween;
a vacuum-resistant membrane extending from the distal end of the catheter body and defining a clot aspiration path contiguous with the aspiration lumen in the catheter body so that applying a vacuum to the proximal end of the aspiration lumen can draw clot into the central clot-receiving passage; and
a distal cap which covers a distal end of the clot aspiration path in a first position and uncovers the distal end of the clot aspiration path in a second position, wherein the distal cap is configured to be removed from the catheter prior to applying the vacuum to the proximal end of the aspiration lumen.

32. The aspiration catheter of claim 31, further comprising a scaffold extending distally from the distal end of the catheter body to radially support the vacuum-resistant membrane.

33. The aspiration catheter of claim 32 wherein at least a distal portion of the scaffold is radially self-expandable from a delivery configuration to an extraction configuration.

34. The aspiration catheter of claim 32, wherein a distal end of the scaffold extends distally beyond a distal end of the vacuum-resistant membrane.

35. The aspiration catheter of claim 32, wherein a distal end of the scaffold and a distal end of the vacuum-resistant membrane terminate about the same location.

36. The aspiration catheter of claim 33, wherein the distal cap is configured to hold the distal portion of the scaffold in the delivery configuration and to release the distal portion of the scaffold to self-expand into the extraction configuration.

37. The aspiration catheter of claim 31, wherein the distal cap is attached to a removable inner elongated tubular body which is configured to be advanced and retracted to covers and uncover the distal end of the clot aspiration path.

38. The aspiration catheter of claim 37, wherein inner elongated tubular body and distal cap are further configured to be withdrawn and removed through the aspiration lumen of the catheter body.

39. The aspiration catheter of claim 33, wherein the distal portion of the scaffold in said extraction configuration engages an inner wall of the blood vessel to substantially prevent blood proximal to the scaffold from entering the clot aspiration path when said vacuum is applied.

40. The aspiration catheter of claim 39, wherein the scaffold in its extraction configuration has a substantially conical region with a proximally oriented apical opening attached to the distal end of the catheter body and a distally oriented open base configured to engage an inner wall of the blood vessel and direct clot into the central clot-receiving passage when the vacuum is applied to a proximal end of the aspiration lumen.

41. The aspiration catheter of claim 40, wherein the cylindrical distal region has a diameter when expanded in a range from 2.2 mm and 5.5 mm and length when expanded in a range from 1 mm and 150 mm.

42. The aspiration catheter of claim 33, wherein the distal portion of the scaffold comprises any one of a sinusoidal ring, a single undulating element, and a serpentine pattern.

43. The aspiration catheter of claim 33, wherein the scaffold comprises a single element following a single path to form a cylindrical or conical envelope.

44. The aspiration catheter of claim 42, wherein the single path is a closed loop.

45. The aspiration catheter of claim 42, wherein the single path is open.

46. The aspiration catheter of claim 33, wherein a distal portion of the scaffold is uncovered and configured to do at least one of invaginate the clot, break the clot, and facilitate extraction of the clot when said distal portion is expanded to facilitate suction of said clot into the aspiration lumen.

47. The aspiration catheter of claim 33, wherein an open port of the distal tip of the scaffold in its extraction configuration has an area which is 1.5 to 10 times greater than the open port area of the aspiration lumen within the fixed diameter catheter body.

48. The aspiration catheter of claim 33, wherein the entire scaffold comprises an expandable distal segment.

49. The aspiration catheter of claim 33, wherein the vacuum-resistant membrane is coupled to at least a distal portion the scaffold.

50. The aspiration catheter of claim 33, wherein the delivery configuration of the distal portion of scaffold is smaller than the distal end of the catheter body.

51. The aspiration catheter of claim 33, wherein an inner surface of the distal portion of scaffold is coated with a lubricious material.

52. The aspiration catheter of claim 33, wherein the scaffold in its extraction configuration is expandable to size in a range from that of the clot to that of the vessel.

53. The aspiration catheter of claim 33, wherein the expandable scaffold comprises one or more features selected from the group consisting of sharp edges, metallic protrusions, fins, hook elements, and slots to improve cutting of or gripping the clot.

54. The aspiration catheter of claim 33, wherein the vacuum-resistant membrane comprises an expandable sleeve which covers the at least first coil to enclose the central clot-receiving passage to create a continuous vacuum path from the aspiration lumen to a distal end of the radially distal expandable segment.

55. The aspiration catheter of claim 54, wherein the expandable sleeve comprises at least one of an elastic section, a folded section, and a furled section.

56. The aspiration catheter of claim 33, wherein the conical region of the scaffold comprises a plurality of struts having proximal ends disposed about the proximally oriented apical opening and distal ends disposed about the distally oriented open base.

57. The aspiration catheter of claim 56, wherein the struts are arranged individually with free proximal ends coupled only by the vacuum-resistant membrane.

58. The aspiration catheter of claim 56, wherein the struts interconnected.

59. The aspiration catheter of claim 56, wherein the struts are arranged in a serpentine pattern with crown regions disposed about both the proximally oriented apical opening and the distally oriented open base.
